(19) United States
(12) Patent Application Publication
(10) Pub. No.: US 2025/0114418 A1
(43) Pub. Date: Apr. 10, 2025

(54) USE OF BOSWELLIA FOR TREATING SARS-COV-2 INFECTION

(71) Applicant: Christoph VON KEUDELL, Tassara (IT)

(72) Inventor: Christoph VON KEUDELL, Tassara (IT)

(21) Appl. No.: 18/290,642

(22) PCT Filed: Jul. 20, 2022
(86) PCT No.: PCT/EP2022/070413

§ 371 Date: Jan. 19, 2024

Related U.S. Application Data
(60) Provisional application No. 63/223,749, filed on Jul. 20, 2021.

(51) Int. Cl. A61K 36/324; A61K 31/19; A61K 31/215; A61K 47/24

(52) U.S. Cl. CPC A61K 36/324; A61K 31/19; A61K 31/215; A61K 47/24

(57) ABSTRACT

The present invention relates to the use of extracts from Boswellia species and pharmaceutical compositions comprising Boswellia extracts in the treatment of SARS-CoV-2 infection and related medical conditions.

### FIELD OF THE INVENTION

The present invention relates to the use of extracts from Boswellia species and pharmaceutical compositions comprising Boswellia extracts in the treatment of SARS-CoV-2 infection and related medical conditions.

### BACKGROUND OF THE INVENTION

The ongoing coronavirus disease 2019 (COVID-19) global pandemic is caused by the severe acute respiratory syndrome coronavirus 2 (SARS-CoV-2; also known as 2019-nCoV or HCoV-19). Together with SARS-CoV-1, identified in 2003, and Middle East respiratory syndrome coronavirus (MERS-CoV), identified in 2012, SARS-CoV-2 belongs to the β-coronavirus genus within the Coronaviridae family. SARS-CoV-2 is an enveloped virus, containing a positive sense single-stranded RNA genome of about 30 kb, which encodes 16 nonstructural proteins (nsp 1-16), 4 structural proteins [spike (S), envelop (E), membrane (M), and nucleocapsid (N)], and 8 accessory proteins (ORF3a, ORF3b, ORF6, ORF7a, ORF7b, ORF8, ORF9b, and ORF10). The nsps are responsible for viral replication, the structural proteins for virion formation, and the accessory proteins facilitate viral infection, but are not essential for viral replication.

The most common symptoms of infection with SARS-CoV-2 initially are fever, dry cough and tiredness. More severe infection of the lower respiratory tract can lead to more serious symptoms, such as difficulty in breathing or shortness of breath and chest pain or pressure. At this point patients may need to be hospitalized and if the oxygen saturation level of the blood is reduced, they will require supplemental oxygen or ventilator support in order to relieve symptoms. Systemic inflammation and serious morbidity or death can follow. Severe SARS-CoV-2 infection causes cytokine release syndrome (CRS), leading to acute respiratory distress syndrome (ARDS), acute kidney and cardiac injury, liver dysfunction, and multiorgan failure.

While most patients infected with SARS-CoV-2 remain asymptomatic or develop only mild symptoms, up to 20% of patients develop severe or critical disease with interstitial pneumonia often accompanied by ARDS, requiring respiratory support and intensive care. It was shown that profound immune dysregulation, in particular cytokine storm (i.e., maladaptive cytokine release in response to infection), may lead to the progression of COVID-19 to critical disease (Kosmaczewska and Frydecka, Pol. Arch. Intern. Med. 2020, 130(9), 779-788). Aside from ARDS, cytokine storm may result in septic shock in a subset of patients with COVID-19, characterized by elevated concentrations of acute phase factors and macrophage activation syndrome, including hepatic dysfunction with hyperferritinemia and diffuse intravascular coagulation.

The severity of COVID-19 is correlating with pathologically increased serum levels of pro-inflammatory cytokines and other factors leading to adverse outcomes including, but not limited to IL-1β, IL-2, IL-5, IL-6, IL-7, IL-8, IL-13, IL-15, IL-18, TNF-α, G-CSF, GM-CSF, MCP-1, CCL2 (chemokine (C—C motif) ligand 2), CCL3 and CCL4. There is mounting evidence that a dysregulated immune system is correlating with COVID-19 patient outcome. Accordingly, control of the cytokine storm in its early stage is key for improvement of treatment and reduction of mortality. However, no treatment or active agent has proven to be effective in this regard so far.

Leukotrienes are important lipid mediators of the inflammatory response and play a critical role in the induction of pathological inflammation and pro-inflammatory cytokine production. Leukotriene biosynthesis is initiated by 5-lipoxygenase (5-LOX). Although several studies have discussed the role of 5-lipoxygenase (5-LOX) in viral infections, such as influenzae and SARS, it remains unexplored in the pathophysiology of COVID-19. 5-LOX acts on free arachidonic acid (AA) to form proinflammatory leukotrienes (LTs).

There is an unmet need for medications that beneficially influence the patient's immune reaction towards infection with SARSCoV-2, in particular medications that are able to suppress cytokine storm to reduce the likelihood of progression to critical disease and to reduce mortality. The immune responses triggered by SARS-CoV-2 are different from those triggered by other coronaviruses (SARS-CoV and MERS-CoV). SARS-CoV-2 infection triggers an increase in the secretion of Th2 cytokines, such as interleukine-4 (IL-4) and IL-10, whereas SARS-CoV causes a decrease in IL-10, for example. Also, the infected host cells and the changes of immune cells triggered by SARS-CoV-2 are different (Liu et al., J. Inflamm. Res. 2021, 14, 5475-5487). This is a clear indication that different treatments are needed for SARS-CoV-2 than for other coronaviruses.

Natural compounds have attracted substantial attention for their potential in the treatment of several inflammatory conditions, including chronic diseases such as arthritis, diabetes, asthma, cancer, inflammatory bowel disease, Parkinson's disease and Alzheimer's. Advantageously, natural compounds are in many cases largely devoid of toxicity and thus often exhibit a favorable therapeutic index, i.e., these compounds possess a wide therapeutic window of doses that show some efficacy without resulting in major toxicity.

Pharmacologically active natural compounds include Boswellia extracts, i.e., extracts from the oleo resin of tree species belonging to the Boswellia genus. Boswellia extracts, which comprise a range of active agents, including a series of pentacyclic triterpene molecules referred to as boswellic acids, have previously been shown to be capable of modulating the immune system and to ameliorate a wide range of inflammatory diseases such as arthritis, diabetes, asthma, cancer, or inflammatory bowel disease.

The present invention addresses an unmet need for immune-modulating medication for SARS-CoV-2 patients that is well-tolerated, efficiently dampens the dysregulated immune system and is at the same time easily accessible without putting a high economic burden on the health systems of countries affected by large-scale SARS-CoV-2 outbreaks.

### SUMMARY OF THE INVENTION

One aspect of the invention provides for a Boswellia extract for use in the treatment or prevention of infection with SARS-CoV-2 or a variant thereof in a subject.

In one embodiment, the Boswellia species is selected from the group consisting of Boswellia sacra, Boswellia carterii, Boswellia frereana, Boswellia serrata (also referred to as B. thurifera) and Boswellia papyrifera.

In a further embodiment, the Boswellia extract comprises at least one active agent, selected from the group consisting of α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) and 3-O-Acetyl-11-keto-β-boswellic acid (AKBA).

In another embodiment, the Boswellia extract comprises at least about 50% by weight of triterpenoid acids.

In a further embodiment, the Boswellia extract is selectively enriched for any one of α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) and 3-O-Acetyl-11-keto-β-boswellic acid (AKBA) or a combination thereof.

In yet another embodiment, the Boswellia extract is selectively enriched for 3-O-Acetyl-11-keto-β-boswellic acid (AKBA).

In a further aspect, the invention provides a pharmaceutical composition comprising the Boswellia extract according to the invention, for use in the treatment or prevention of infection with SARS-CoV-2 or a variant thereof in a subject. Preferably, the Boswellia extract amounts for more than 80% (w/w) of all active ingrediedients of pharmaceutical composition; more preferably more than 85% (w/w); and most preferably for more than 90% (w/w). In particular, the pharmaceutical composition may be free from glycyrrhizin.

In one embodiment, the Boswellia extract or pharmaceutical composition is administered via feeding tube.

In a specific embodiment, the pharmaceutical composition comprises soybean lecithin, rapeseed lecithin, sunflower lecithin, egg lecithin or a mixture thereof.

In one embodiment, the lecithin content in the composition is at least about 20% (w/w).

In a further embodiment, the Boswellia extract or pharmaceutical composition is administered via transdermal patch.

In yet another embodiment, the daily dose is from about 100 to about 16 000 mg Boswellia extract.

In one embodiment, the subject
a) exhibits one or more risk factor(s), selected from the group consisting of older age and pre-existing conditions such as obesity, hypertension, cardiovascular disease, diabetes mellitus, chronic lung disease, cancer and cerebrovascular disease; and/or
b) carries at least one genotypic variant associated with increased risk of SARS-CoV-2 infection, hospitalization or death.

In a further embodiment, the Boswellia extract or pharmaceutical composition inhibits activation of NF-κB, downregulates TNFα, decreases the level of IL-1, IL-2, IL-4, IL-6, IL-8, IL-12 and/or IFN-γ, inhibits iNOS, inhibits the formation of oxygen radicals and activation of proteases, inhibits COX-2 and/or inhibits 5-LOX.

In yet another embodiment, the Boswellia extract or pharmaceutical composition prevent, suppress, alleviate or reduce cytokine storm in a subject.

A recent study, published only after the priority date of this application, underlines that Boswellia extracts are potential therapeutic agents for SARS-CoV-2 patients. The active pharmaceutical ingredients modulate the immune response of a SARS-CoV2 infection inhibiting the secretion of pro-inflammatory cytokines (Tond et al., Inflammopharmacology 2022, 30, 465-475).

### DETAILED DESCRIPTION OF THE INVENTION

The invention relates to Boswellia extract or pharmaceutical compositions comprising Boswellia extract for use in the treatment or prevention of infection with SARS-CoV-2 or a variant thereof in a subject.

Boswellia, also referred to as frankincense or olibanum-tree, is a moderate-to-large branching tree belonging to the Burseraceae family that grows in high, dry habitats in India, Northern Africa, and the Middle East. Its gummy resin is tapped from the tree by means of incisions in its bark that allow the thick, oily substance to ooze out for collection and drying.

The invention generally relates to extracts from the oleo resin of any species belonging to the Boswellia genus, pharmaceutical compositions comprising said extracts and uses thereof. In a preferred embodiment, the Boswellia species is selected from the group consisting of Boswellia sacra, Boswellia carterii, Boswellia frereana, Boswellia serrata (also referred to as B. thurifera) and Boswellia papyrifera. In a more preferred embodiment, the Boswellia species is selected from the group consisting of Boswellia sacra, Boswellia serrata and Boswellia papyrifera, most preferably the Boswellia species is Boswellia sacra or Boswellia serrata. In a specific embodiment, the Boswellia species is Boswellia sacra. In a further specific embodiment, the Boswellia species is Boswellia carterii. In a further specific embodiment, the Boswellia species is Boswellia frereana. In a further specific embodiment, the Boswellia species is Boswellia serrata. In a further specific embodiment, the Boswellia species is Boswellia papyrifera.

The Boswellia oleo resin, which is thick and oily, is commonly extracted with organic, aqueous or mixed organic-aqueous solvents to generate a Boswellia extract that can be used as described herein. In the context of the present invention the term “Boswellia extract” refers to a mixture of components which are present in the oleo resin of Boswellia species and which are soluble in a suitable solvent. The solvent may be an aqueous solvent or an organic solvent, preferably an organic solvent, or a mixture of organic solvents. The solvent may be a polar solvent, typically an alcohol such as methanol or ethanol or a nonpolar solvent such as hexane. Preferred examples of suitable solvents include methanol, ethanol, hexane, ethyl acetate, diethyl ether, chloroform, methylene chloride, petroleum ether, acetone, pentane, toluene, or a mixture thereof. Particularly preferred solvents include methanol, ethanol, ethyl acetate, and chloroform, or a mixture thereof. Other suitable solvents will be well known to those skilled in the art of plant component extraction.

In a specific embodiment, the solvent is methanol. In a specific embodiment, the solvent is ethanol. In a specific embodiment, the solvent is ethyl acetate. In a specific embodiment, the solvent is chloroform.

The extract may be obtained by mixing the gum resin with any of the abovementioned solvents and extracting for a suitable period of time, for example several days, in a light-free environment so that the soluble components of the gum are extracted into the solvent to remove any insoluble resin. Extraction may be performed at temperatures ranging from about 5° C. to about 150° C., preferably about 10° C. to about 100° C., about 15° C. to about 75° C. or about 20° C. to about 50° C. More preferably, extraction is performed at temperatures ranging from about 20° C. to about 50°, such as about 20° C., about 25° C., about 30° C., about 35° C., about 40° C., about 45° C. or about 50° C. The solvent may then be removed to obtain the extract.

Alternatively, the extract may be obtained by steam distillation of the Boswellia tree oleo resin.

A further typical procedure for preparing a Boswellia extract includes a) providing crude Boswellia (e.g., Boswellia serrata) gum resin, b) extracting the resin with a water-immiscible organic solvent as disclosed herein (e.g., ethyl acetate or chloroform) and discarding the insoluble gum substances separated by filtration, c) repeatedly washing the organic extract with an aqueous solution to remove the acid compounds, wherein the aqueous solution may be an acidic, basic, or a buffered pH neutral solution, d) successive washing of the organic extract with water and saline, e) evaporation of the organic layer in vacuo at elevated temperature (e.g., 60-70° C.) to give an oil residue, f) subsequent removal of the volatile components from the abovementioned oil residue under high vacuum and high temperature conditions to yield a Boswellia extract in the form of a viscous oil.

Alternatively, Boswellia extract can also be prepared by a method comprising: a) preparation of alcohol or water-alcohol extract of gum-resin Boswellia (e.g., Boswellia serrata), b) distributing the alcoholic extract between an aqueous solution and a water-immiscible organic solvent as disclosed herein (e.g., ethyl acetate or chloroform), wherein the aqueous solution may be an acidic, basic, or a buffered pH neutral solution, c) separating the organic layer, followed by obtaining an oil residue by evaporating the organic layer in vacuo at elevated temperature (e.g., 60-70° C.), d) subsequent removal of the volatile components from the above-mentioned oil residue under high vacuum and high temperatures to give a Boswellia extract in the form of a viscous oil.

The Boswellia extract according to the invention comprises a range of active agents that mediate the anti-inflammatory action of the inventive extracts and pharmaceutical compositions comprising the extracts. These active agents include boswellic acids, such as α-boswellic acid, β-boswellic acid, and derivatives thereof, including 3-O-Acetyl-α-boswellic acid, 3-O-Acetyl-11-keto-α-boswellic acid, 3-O-Acetyl-β-boswellic acid (AcBA), 11-keto-β-boswellic acid (KBA) and 3-O-Acetyl-11-keto-β-boswellic acid (AKBA). Further active agents comprised in Boswellia extract include epi-lupeol, β-amyrin, α-amyrin, α-phellandrene dimers, α-thujene and α-phellandrene and their isomers. Without being bound to a theory, it is currently believed that all those ingredients are relevant for the pharmaceutical activity of the composition, as outlined more specifically elsewhere herein.

Advantageously, the Boswellia extract comprises at least one active agent, selected from the group consisting of α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) and 3-O-Acetyl-11-keto-β-boswellic acid (AKBA). In one embodiment, the Boswellia extract comprises α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) and 3-O-Acetyl-11-keto-β-boswellic acid (AKBA). In a preferred embodiment, the Boswellia extract comprises α-boswellic acid, β-boswellic acid, 3-O-Acetyl-β-boswellic acid (AcBA), 11-Keto-β-boswellic acid (KBA) and 3-O-Acetyl-11-keto-β-boswellic acid (AKBA). In a further preferred embodiment, the Boswellia extract comprises α-boswellic acid, β-boswellic acid, 11-Keto-β-boswellic acid (KBA) and 3-O-Acetyl-11-keto-β-boswellic acid (AKBA). In a further preferred embodiment, the Boswellia extract comprises 11-Keto-β-boswellic acid (KBA) and 3-O-Acetyl-11-keto-β-boswellic acid (AKBA). In a further preferred embodiment, the Boswellia extract comprises 3-O-Acetyl-11-keto-β-boswellic acid (AKBA).

The Boswellia oleo resin, obtained by incision of the bark, is composed by essential oil (about 5-9%), mucopolysaccarides (about 21-22%), and pure resin (about 65-85%), containing tetracyclic and pentacyclic triterpene acids, of which boswellic acids (BAs) are the most important bioactive molecules.

Preferably, the Boswellia extract according to the invention comprises at least about 50%, at least about 60%, at least about 70%, at least about 75%, at least about 80%, at least about 85%, at least about 90%, or at least about 95% by weight of triterpenoid acids (i.e., boswellic acid compounds). In one particularly preferred embodiment, the Boswellia extract according to the invention comprises at least about 50% by weight of triterpenoid acids. In another particularly preferred embodiment, the Boswellia extract according to the invention comprises at least about 60% by weight of triterpenoid acids. In one particularly preferred embodiment, the Boswellia extract according to the invention comprises at least about 70% by weight of triterpenoid acids. In one particularly preferred embodiment, the Boswellia extract according to the invention comprises at least about 75% by weight of triterpenoid acids. In one particularly preferred embodiment, the Boswellia extract according to the invention comprises at least about 80% by weight of triterpenoid acids. In one particularly preferred embodiment, the Boswellia extract according to the invention comprises at least about 85% by weight of triterpenoid acids. In one particularly preferred embodiment, the Boswellia extract according to the invention comprises at least about 90% by weight of triterpenoid acids. In one particularly preferred embodiment, the Boswellia extract according to the invention comprises at least about 95% by weight of triterpenoid acids. Boswellic acid content or triterpenoid content is commonly determined by high performance liquid chromatography (HPLC), optionally coupled with mass spectrometry (HPLC-MS).

The content of acetylated and non-acetylated boswellic acids can vary, depending on the species or variety of Boswellia that was used to obtain the oleo resin to be extracted. In one embodiment of the Boswellia extract according to the invention, the ratio of acetylated to non-acetylated boswellic acids is about 5, about 4, about 3, about 2, about 1, about 0.5, about 0.33, about 0.25, or about 0.125. In a specific embodiment, the ratio of acetylated to non-acetylated boswellic acids is >1 (i.e., above 1). In a further specific embodiment, the ratio of acetylated to non-acetylated boswellic acids is <1 (i.e., below 1).

The Boswellia extract according to the invention may be selectively enriched for one or more active agent that naturally occurs in Boswellia extract. As used herein, selective enrichment (or “selectively enriched”) refers to enrichment relative to natural Boswellia extract, i.e., relative to Boswellia extract which has not undergone selective enrichment. Obtaining the Boswellia extract as such from crude Boswellia oleo resin is not included by the term selective enrichment (or “selectively enriched”). Selective enrichment may be performed using methods known in the art, including extraction, distillation, crystallisation, filtration, chromatography (e.g., high-performance liquid chromatography) or a combination of these methods.

For example, the Boswellia extract according to the invention may be selectively enriched for any one of α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) or 3-O-Acetyl-11-keto-β-boswellic acid (AKBA), or a combination thereof. In one embodiment, the Boswellia extract is selectively enriched for α-boswellic acid. In a further embodiment, the Boswellia extract is selectively enriched for β-boswellic acid. In a further embodiment, the Boswellia extract is selectively enriched for 3-O-Acetyl-α-boswellic acid. In a further embodiment, the Boswellia extract is selectively enriched for 3-O-Acetyl-11-keto-α-boswellic acid. In a further embodiment, the Boswellia extract is selectively enriched for 3-O-Acetyl-β-boswellic acid (AcBA). In a further embodiment, the Boswellia extract is selectively enriched for 11-Keto-β-boswellic acid (KBA). In a further embodiment, the Boswellia extract is selectively enriched for 3-O-Acetyl-11-keto-β-boswellic acid (AKBA).

It has surprisingly been found that these boswellic acids are particularly beneficial for the treatment of Covid-19-related inflammatory conditions and pain related to these conditions. 3-O-Acetyl-11-keto-β-boswellic acid (AKBA) and 11-Keto-β-boswellic acid (KBA) show a particularly high anti-inflammatory response. Without wishing to be bound by theory, it is currently believed that they strongly interfere with leukotriene synthesis. Further physiological effects that can be attributed to specific boswellic acids and that are associated with Covid-19 comprise cytokine inhibition as well as immunomodulation (3-O-Acetyl-α-boswellic acid, 11-Keto-β-boswellic acid (KBA) and 3-O-Acetyl-11-keto-β-boswellic acid (AKBA)); anti-bacterial activities (α-boswellic acid and β-boswellic acid); improvement of cognitive impairments (α-boswellic acid and 3-O-Acetyl-11-keto-β-boswellic acid (AKBA)); improvement of inflammatory bowel diseases, ulcerative colitis as well as gastroprotective activities (α-boswellic acid and 3-O-Acetyl-11-keto-β-boswellic acid (AKBA)); cardioprotective activities and treatment of myocarditis (3-O-Acetyl-11-keto-β-boswellic acid (AKBA); and neuroprotective activities (3-O-Acetyl-11-keto-β-boswellic acid (AKBA). Boswellia extracts containing the aforementioned boswellic acids, in particular Boswellia extracts selectively enriched for any single of the aforementioned boswellic acids or any combination of one or more of the aforementioned boswellic acids, may beneficially be used for the treatment of the aforementioned conditions associated with Covid-19. Conditions associated with Covid-19 include conditions that persist beyond the period of viral infection (long-term effects), particularly conditions associated with long-Covid-19, post-acute-Covid-19 or chronic post-Covid-19. Such long-term effects comprise cardiovascular impairments, cognitive impairments, gastrointestinal impairments and physical organ damage of the heart, blood vessels, brain, nervous system, liver, lung, intestine or kidney.

In preferred embodiments, the Boswellia extract is selectively enriched for α-boswellic acid and 3-O-Acetyl-11-keto-β-boswellic acid (AKBA). In further preferred embodiments, the Boswellia extract is selectively enriched for β-boswellic acid and 3-O-Acetyl-11-keto-β-boswellic acid (AKBA). In further preferred embodiments, the Boswellia extract is selectively enriched for 3-O-Acetyl-α-boswellic acid and 3-O-Acetyl-11-keto-β-boswellic acid (AKBA). In further preferred embodiments, the Boswellia extract is selectively enriched for 3-O-Acetyl-β-boswellic acid (AcBA) and 3-O-Acetyl-11-keto-β-boswellic acid (AKBA). In further preferred embodiments, the Boswellia extract is selectively enriched for 11-Keto-β-boswellic acid (KBA) and 3-O-Acetyl-11-keto-β-boswellic acid (AKBA).

Further, the active agent(s) may include cembranoids (fourteen-membered carbocyclic rings with an isopropyl residue at position Cl and three methyl groups at positions C4, C8 and C12), and derivatives thereof.

Cembranoids may comprise (i) a tetrahydrofuran structure formed by cyclization between Cl and C12 through an ether bond, in particular (1S,4R,11S,12R)-1:12-epoxy-11-hydroxy-cembra-7E-en-3-one (Sacraoxide C). Specific examples of cembranoids according to item (i) include (a) a trisubsituted trans-olefin at the C3/4 position of the original cembranoid structure, in particular (1R,2R,5E,9Z)-1,5,9-trimethyl-12-propan-2-yl-15-oxabicyclo[10.2.1]pentadeca-5,9-dien-2-ol (Incensole) and (1R,2R,6S,8R,13S,Z)-13-isopropyl-1,10-dimethyl-5-methylene-7,16-dioxatricyclo[11.2.1.0⁶,8]hexadec-10-en-2-ol (Boscartin AX). Specific examples of cembranoids according to item (a) include (aa) an O-Acetyl group at C11, in particular [(1R,2S,5E,9E,12S)-1,5,9-trimethyl-12-propan-2-yl-15-oxabicyclo[10.2.1]pentadeca-5,9-dien-2-yl]acetate (Incensole acetate). Specific examples of cembranoids according to item (aa) include (aaa) a carbonyl group at the C6 and/or C9 position, in particular (1S,11S,12R)-1:12-epoxy-11-acetoxy-cembra-3E,7E-dien-6-one (Sacraoxide E) and (1S,11S,12R)-1:12-epoxy-11-acetoxy-cembra-3E,7E-dien-9-one (Sacraoxide F).

Further, cembranoids may comprise (ii) a hydroxy group at the C1 and/or C5 position of the original cembranoid structure, in particular (1S,5R,7E,11E)-1-isopropyl-8,12-dimethyl-4-methylenecyclotetradeca-7,11-diene-1,5-diol.

It has surprisingly been found that cembranoids are particularly beneficial for the treatment of Covid-19-related inflammatory conditions, for the recovery of neurobehavioral and cognitive functions (Incensole and Incensole acetate) and for hepaprotective activity (Boscartin AX).

Any of the abovementioned active agents or combination thereof may be selectively enriched at least about 2-fold, at least about 3-fold, at least about 4-fold, at least about 5-fold, at least about 10-fold, at least about 50-fold or at least about 100-fold relative to natural Boswellia extract, i.e., relative to Boswellia extract that has not undergone selective enrichment as described herein. In one embodiment, the active agent or combination thereof is selectively enriched at least about 2-fold relative to natural Boswellia extract. In a further embodiment, the active agent or combination thereof is selectively enriched at least about 3-fold relative to natural Boswellia extract. In a further embodiment, the active agent or combination thereof is selectively enriched at least about 4-fold relative to natural Boswellia extract. In a further embodiment, the active agent or combination thereof is selectively enriched at least about 5-fold relative to natural Boswellia extract. In a further embodiment, the active agent or combination thereof is selectively enriched at least about 10-fold relative to natural Boswellia extract. In a further embodiment, the active agent or combination thereof is selectively enriched at least about 50-fold relative to natural Boswellia extract. In a further embodiment, the active agent or combination thereof is selectively enriched at least about 100-fold relative to natural Boswellia extract. Accordingly, in one embodiment, the Boswellia extract of the invention comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) α-boswellic acid. In a further embodiment, the Boswellia extract according to the invention comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) β-boswellic acid. In a further embodiment, the Boswellia extract according to the invention comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 3-O-Acetyl-α-boswellic acid. In a further embodiment, the Boswellia extract according to the invention comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 3-O-Acetyl-11-keto-α-boswellic acid. In a further embodiment, the Boswellia extract according to the invention comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 3-O-Acetyl-boswellic acid (AcBA). In a further embodiment, the Boswellia extract according to the invention comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 11-Keto-β-boswellic acid (KBA). In a further embodiment, the Boswellia extract according to the invention comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 3-O-Acetyl-11-keto-β-boswellic acid (AKBA).

In a particularly preferred embodiment, the Boswellia extract according to the invention comprises at least about 1% (w/w) 3-O-Acetyl-11-keto-β-boswellic acid (AKBA). In a further particularly preferred embodiment, the Boswellia extract according to the invention comprises at least about 2% (w/w) 3-O-Acetyl-11-keto-β-boswellic acid (AKBA). In a further particularly preferred embodiment, the Boswellia extract according to the invention comprises at least about 3% (w/w) 3-O-Acetyl-11-keto-β-boswellic acid (AKBA). In a further particularly preferred embodiment, the Boswellia extract according to the invention comprises at least about 4% (w/w) 3-O-Acetyl-11-keto-β-boswellic acid (AKBA). In a further particularly preferred embodiment, the Boswellia extract according to the invention comprises at least about 5% (w/w) 3-O-Acetyl-11-keto-β-boswellic acid (AKBA). In a further particularly preferred embodiment, the Boswellia extract according to the invention comprises at least about 10% (w/w) 3-O-Acetyl-11-keto-β-boswellic acid (AKBA). In a further particularly preferred embodiment, the Boswellia extract according to the invention comprises at least about 15% (w/w) 3-O-Acetyl-11-keto-β-boswellic acid (AKBA). In a further particularly preferred embodiment, the Boswellia extract according to the invention comprises at least about 20% (w/w) 3-O-Acetyl-11-keto-β-boswellic acid (AKBA). In a further particularly preferred embodiment, the Boswellia extract according to the invention comprises at least about 25% (w/w) 3-O-Acetyl-11-keto-β-boswellic acid (AKBA). In a further particularly preferred embodiment, the Boswellia extract according to the invention comprises at least about 50% (w/w) 3-O-Acetyl-11-keto-β-boswellic acid (AKBA). In a further particularly preferred embodiment, the Boswellia extract according to the invention comprises at least about 75% (w/w) 3-O-Acetyl-11-keto-β-boswellic acid (AKBA). It has surprisingly been found that AKBA is particularly beneficial for the treatment of Covid-19 related cognitive impairments and chronic inflammatory conditions. These impairments and conditions include impairments and conditions that persist beyond the period of viral infection (long-term effects), particularly impairments and conditions associated with long-Covid-19, post-acute-Covid-19 or chronic post-Covid-19. Such long-term effects comprise cardiovascular impairments, cognitive impairments, gastrointestinal impairments and physical organ damage of the heart, blood vessels, brain, nervous system, liver, lung, intestine or kidney.

In one aspect, the invention relates to pharmaceutical compositions comprising the Boswellia extract of the invention and uses thereof. The pharmaceutical compositions of the invention may comprise any components of the Boswellia extract in any of the amounts disclosed herein. Preferably, the Boswellia extract amounts for more than 80% (w/w) of all active ingredients of pharmaceutical composition; more preferably more than 85% (w/w); and most preferably for more than 90% (w/w). In particular, the pharmaceutical composition may be free from glycyrrhizin.

The pharmaceutical composition of the invention can be suitably formulated in a manner known to the skilled artisan, optionally using one or more pharmaceutically acceptable carriers or diluents. Suitable diluents are polyglycols, ethanol, water and buffer solutions. Suitable buffer substances include N,N-dibenzylethylenediamine, diethanolamine, ethylenediamine, N-methylglucamine, N-benzylphenethylamine, diethylamine, phosphate, sodium bicarbonate and sodium carbonate. However, it is also possible to work without diluent.

The pharmaceutical composition of the invention may be suitable for (i.e., formulated for) administration by inhalation, topical, oral, parenteral, intravenous, intraarterial, intraperitoneal, intramuscular, subcutaneous, rectal or vaginal administration. The pharmaceutical composition may be suitable for administration by injection, preferably for intravenous or intraarterial injection or drip.

The Boswellia extract or pharmaceutical composition of the invention may be used for the treatment of the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabilizer. Instead of the propellant, compressed air can also be used, it being possible for this to be produced as required by means of a suitable compression and expansion device.

A further preferred pharmaceutical composition of the invention is formulated as a tablet, capsule or pill, which are prepared by methods known in the art with pharmaceutically acceptable diluents, such as binders (for example, pregelatinized corn starch, polyvinylpyrrolidone or hydroxypropylmethyl cellulose), fillers (for example, lactose, sucrose, mannitol, corn starch, microcrystalline cellulose or calcium hydrogen phosphate); lubricants (for example, stearic acid, polyethylene glycol, magnesium stearate, talc or silicon dioxide); disintegrating agents (for example, potato starch, sodium starch glycolate or sodium carboxymethylcellulose); or wetting agents (for example, sodium lauryl sulfate). The tablet, capsule or pill may be coated by methods known in the art.

Liquid preparations for oral administration may be in the form of, for example, aqueous or oily solutions, syrups, elixirs, emulsions or suspensions, or they may be in the form of a dry product for constitution with water or other suitable carrier prior to use. Such liquid preparations may be prepared by methods known in the art with pharmaceutically acceptable additives, such as suspending agents (for example, sorbitol syrup, cellulose derivatives, glucose/sugar syrup, gelatin, aluminum stearate gel, or hydrated edible fats); emulsifying agents (for example, lecithin, gum arabic or sorbitan monooleate); non-aqueous carriers (for example, almond oil, oily esters, ethyl alcohol or fractionated vegetable oils); and preservatives (for example, methyl or propyl p-hydroxybenzoates or sorbic acid). The liquid preparations may also contain known buffers, flavoring agents, colorants and sweeteners, as required.

The pharmaceutical composition of the invention may be an immediate release formulation, an extended release formulation (also referred to as sustained release formulation) or a formulation for pulsed release. In one embodiment, the pharmaceutical composition is formulated for immediate release. In a further embodiment, the pharmaceutical composition is formulated for extended release or sustained release. As used herein, sustained or extended release refers to release of an active agent or mixture of active agents over a period of about 8 to about 72 h, about 12 to about 48 h or about 16 to about 24 h. In a further embodiment, the pharmaceutical composition is formulated for pulsed release. A pulsed release formulation is capable of providing one or more immediate release pulses at predetermined lag times or at specific sites.

In a particularly preferred embodiment, the Boswellia extract or pharmaceutical composition is formulated to be suitable for administration via feeding tube. Suitable formulations for administration via feeding tube, e.g., lipid-based formulations are known to the skilled artisan. In some embodiments, the formulation comprises 5% (w/w) glucose. The inventors have observed that high-lipid formulations increase bioavailability of the Boswellia extract and the active agents comprised therein. Various fats and/or lecithins may be used, including, but not limited to soybean lecithin, rapeseed lecithin, sunflower lecithin, egg lecithin and the like, vegetable oils, such as avocado oil, almond oil, soybean oil, cottonseed oil, sunflower oil, corn oil, rice oil, palm oil, perilla oil, sesame oil, linseed oil, rapeseed oil, bran oil, olive oil, cacao fat, grape seed oil, peanut oil, coconut oil and the like or animal oils, such as fish oil, chicken oil, beef tallow, pig fat and sheep fat; milk fat such as butter oil, butter, margarine, and the like; and processed oils and fats.

A preferred base for the formulation is lecithin, which facilitates the uptake of active agents comprised in Boswellia extract, thus increasing bioavailability. Thus, in a preferred embodiment, the pharmaceutical composition comprises lecithin, e.g., soybean lecithin, rapeseed lecithin, sunflower lecithin, egg lecithin or a mixture thereof. In a specific embodiment, the formulation comprises soybean lecithin. In a further specific embodiment, the formulation comprises rapeseed lecithin. In a further specific embodiment, the formulation comprises sunflower lecithin. In a further specific embodiment, the formulation comprises egg lecithin.

Preferably, the lipid and/or lecithin content in the pharmaceutical composition is at least about 20% (w/w), at least about 30% (w/w), at least about 40% (w/w), at least about 50% (w/w), at least about 60% (w/w) or at least about 70% (w/w). In a specific embodiment, the lipid content in the pharmaceutical composition is at least about 20% (w/w), at least about 30% (w/w), at least about 40% (w/w), at least about 50% (w/w), at least about 60% (w/w) or at least about 70% (w/w). In a specific embodiment, the lecithin content in the pharmaceutical composition is at least about 20% (w/w), at least about 30% (w/w), at least about 40% (w/w), at least about 50% (w/w), at least about 60% (w/w) or at least about 70% (w/w).

In a highly preferred embodiment, the lecithin content in the pharmaceutical composition is at least about 20% (w/w). In a further highly preferred embodiment, the lecithin content in the pharmaceutical composition is at least about 30% (w/w). In a further highly preferred embodiment, the lecithin content in the pharmaceutical composition is at least about 40% (w/w). In a further highly preferred embodiment, the lecithin content in the pharmaceutical composition is at least about 50% (w/w). In a further highly preferred embodiment, the lecithin content in the pharmaceutical composition is at least about 60% (w/w). In a further highly preferred embodiment, the lecithin content in the pharmaceutical composition is at least about 70% (w/w).

The Boswellia extract or pharmaceutical composition of the invention is administered as necessary. A major advantage of using Boswellia extract is its low toxicity, so that the dosage can be varied by the physician depending on the severity of the disease and the duration of the treatment. In human application, the pharmaceutical composition according to the invention is administered in a dosage of about 100 to about 4000 mg Boswellia extract, preferably about 500 to about 2500 mg Boswellia extract, more preferably about 750 to about 1500 mg Boswellia extract.

The dosages disclosed herein can be administered one to four times daily. Accordingly, in one embodiment, the Boswellia extract or pharmaceutical composition of the invention is administered once daily. In a further embodiment, the Boswellia extract or pharmaceutical composition of the invention is administered twice daily. In a further embodiment, the Boswellia extract or pharmaceutical composition of the invention is administered three times daily. In a further embodiment, the Boswellia extract or pharmaceutical composition of the invention is administered four times daily. In yet another embodiment, the Boswellia extract or pharmaceutical composition of the invention is administered continuously.

The daily dose may range from about 100 to about 16 000 mg, about 500 to about 10 000 mg or about 750 to about 6 000 mg Boswellia extract. In a specific embodiment, the daily dose is about 250 mg Boswellia extract. In a further specific embodiment, the daily dose is about 500 mg Boswellia extract. In a further specific embodiment, the daily dose is about 750 mg Boswellia extract. In a further specific embodiment, the daily dose is about 1 000 mg Boswellia extract. In a further specific embodiment, the daily dose is about 1 500 mg Boswellia extract. In a further specific embodiment, the daily dose is about 2 000 mg Boswellia extract. In a further specific embodiment, the daily dose is about 2 500 mg Boswellia extract. In a further specific embodiment, the daily dose is about 3 000 mg Boswellia extract. In a further specific embodiment, the daily dose is about 3 500 mg Boswellia extract. In a further specific embodiment, the daily dose is about 4 000 mg Boswellia extract. In a further specific embodiment, the daily dose is about 5 000 mg Boswellia extract. In a further specific embodiment, the daily dose is about 6 000 mg Boswellia extract.

In a particularly preferred embodiment, the Boswellia extract or pharmaceutical composition of the invention are administered via feeding tube. The inventors have found that administration of compositions comprising Boswellia extract at higher than common doses leads to efficient reduction of inflammatory processes commonly leading fibrosis in the lung and further organs. Administration via feeding tube was observed to facilitate the uptake of such high doses of Boswellia extract. The feeding tube may be a nasogastric feeding tube, a nasojejunal feeding tube, a gastric feeding tube, a gastrojejunal feeding tube or a jejunal feeding tube. In a preferred embodiment, the feeding tube is a gastric feeding tube.

Accordingly, in a particularly preferred embodiment, the Boswellia extract or pharmaceutical composition of the invention is administered via feeding tube. Administration via gastric feeding tube is particularly suited for administration of high doses of the Boswellia extract and pharmaceutical composition according to the invention. Thus, in a preferred embodiment, the Boswellia extract or pharmaceutical composition of the invention is administered via feeding tube at any one of the daily doses disclosed herein. In a specific preferred embodiment, the Boswellia extract or pharmaceutical composition of the invention is administered continuously via feeding tube, e.g., via gastric feeding tube.

The usually observed low bioavailability of Boswellia extracts and the active agents comprised therein may also be overcome by transdermal administration, in particular by transdermal administration via transdermal patch. The use of transdermal patches leads to efficient uptake and high bioavailability of active agents comprised in Boswellia extract. Moreover, transdermal patches can result in gradual continuous uptake of active agents, which supports the therapeutic effects of the inventive Boswellia extracts and pharmaceutical compositions. Accordingly, in one embodiment, the Boswellia extract or pharmaceutical composition are administered via transdermal patch.

A transdermal patch typically comprises a backing layer impermeable to the drug, an adhesive, which serves to adhere the components of the patch together along with adhering the patch to the skin, and a liner. The liner is removed prior to use of the transdermal patch. Optionally, the transdermal patch comprises a membrane, which controls the release of the drug from the patch. In some embodiments, the transdermal patch comprises a permeation enhancer, i.e., a permeation promoter for the active agents comprised in the patch. Suitable membranes, permeation enhancers and further optional additives, such as stabilizers, matrix fillers, preservatives, etc. are known to the skilled artisan.

The transdermal patch may comprise the Boswellia extract in any of the forms or amounts described herein. In one embodiment, the transdermal patch comprises the Boswellia extract in an amount from about 100 to about 16 000 mg, about 500 to about 10 000 mg or about 750 to about 6 000 mg. In a specific embodiment, the transdermal patch comprises an amount of about 250 mg Boswellia extract. In a further specific embodiment, the transdermal patch comprises an amount of about 500 mg Boswellia extract. In a further specific embodiment, the transdermal patch comprises an amount of about 750 mg Boswellia extract. In a further specific embodiment, the transdermal patch comprises an amount of 1 000 mg Boswellia extract. In a further specific embodiment, the transdermal patch comprises an amount of about 1 500 mg Boswellia extract. In a further specific embodiment, the transdermal patch comprises an amount of about 2 000 mg Boswellia extract. In a further specific embodiment the transdermal patch comprises an amount of about 2 500 mg Boswellia extract. In a further specific embodiment, the transdermal patch comprises an amount of about 3 000 mg Boswellia extract. In a further specific embodiment, the transdermal patch comprises an amount of 3 500 mg Boswellia extract. In a further specific embodiment, the transdermal patch comprises an amount of 4 000 mg Boswellia extract. In a further specific embodiment, the transdermal patch comprises an amount of 5 000 mg Boswellia extract. In a further specific embodiment, the transdermal patch comprises an amount of about 6 000 mg Boswellia extract.

In one embodiment, the transdermal patch is a single-layer drug-in-adhesive type patch. The drug-in-adhesive layer comprises the active agent in admixture with an adhesive, resulting in a drug-in-adhesive layer with defined drug release characteristics. In a further embodiment, the transdermal patch is a multi-layer drug-in-adhesive type patch. A multi-layer drug-in-adhesive type patch comprises at least two layers of drug-in-adhesive with different release properties, optionally separated by a membrane. For example, one layer may be for immediate release and one or more layers may be for rate-controlled release. In a further embodiment, the transdermal patch is a reservoir type patch. A reservoir type patch typically has a separate drug layer (i.e., a layer comprising the active agent), unlike the single-layer and multi-layer drug-in-adhesive systems. The drug layer is a compartment containing a drug solution, suspension or gel separated by the adhesive layer. Optionally, the reservoir type patch comprises a rate-controlling membrane, e.g., made of a polymer like vinyl acetate on a surface between the drug layer and the skin of the subject.

During SARS-CoV-2 infection, capillaries are often occluded, due to the severe inflammation and fibrosis (e.g., pulmonary fibrosis) caused by the viral infection, which reduces drug delivery via other routes. Moreover, COVID-19 is often associated with hepatic complications, even liver failure in severely affected subjects, which may also affect bioavailability of therapeutic agents. Transdermal delivery may overcome these restrictions, as transdermal delivery bypasses the intestinal-hepatic circulation. In the context of the present invention, these advantages arise not only, but in particular when the transdermal patch is applied to the breast of a subject.

Large area transdermal patches were observed to facilitate uptake of higher doses of Boswellia extract and the active agents comprised therein. The transdermal patches according to the invention may have a surface area of about 10 cm², about 15 cm², about 20 cm², about 25 cm², about 30 cm², about 35 cm², about 40 cm², about 45 cm², about 50 cm², about 60 cm², about 70 cm², about 80 cm², about 90 cm², about 100 cm², about 150 cm², about 200 cm², about 300 cm², about 400 cm², about 500 cm², about 600 cm² or about 700 cm². Preferably, the transdermal patch has a surface area of about 100 cm², about 150 cm², about 200 cm², about 300 cm², about 400 cm² or about 500 cm². In a specific embodiment, the transdermal patch has a surface area of about 100 cm². In a further specific embodiment, the transdermal patch has a surface area of about 150 cm². In a further specific embodiment, the transdermal patch has a surface area of about 200 cm². In a further specific embodiment, the transdermal patch has a surface area of about 300 cm². In a further specific embodiment, the transdermal patch has a surface area of about 400 cm². In a further specific embodiment, the transdermal patch has a surface area of about 500 cm². “Surface area” refers to the area of the transdermal patch that is in contact with the skin of the subject during use of the transdermal patch.

The Boswellia extract of the invention may be administered in combination with further active agents or combinations thereof. Non-limiting examples of such active agents include extracts of or preparations obtained from Phyllanthus (e.g., Phyllanthus amarus), Ocimum sanctum, Andrographis panniculata, Withania somnifera, Tinosporia cordifolia, Emblica officinalis, or combinations thereof.

In one embodiment, the Boswellia extract or pharmaceutical composition of the invention is administered in combination with an extract of Phyllanthus, in particular with an extract of Phyllanthus amarus (e.g., Rhyllanthus amarus Schumach et Thonn). Extracts of Phyllanthus comprise anti-fibrotic active substances which can increase uptake of further active agents, such as the inventive Boswellia extract and the active agents comprised therein. This in particular applies to subjects suffering from COVID-19, which is known to lead to fibrosis with a potential negative impact on uptake of active agents.

The pharmaceutical composition of the invention can be used for the treatment of prevention of Coronavirus infection, in particular for the treatment or prevention of an infection with SARS-CoV-2 or a variant thereof. For example, the SARS-CoV-2 variant may be selected from the group consisting of AV.1, AY.1, AY.2, A.23.1, B.1.1.7, B1.1.318, B.1.351, B.1.351.1, B.1.351.2, B.1.351.3, B.1.427, B.1.429, B.1.525, B.1.526, B.1.526.1, B.1.526.2, B.1.617, B.1.617.1, B.1.617.2, B.1.617.3, C.36.3, C.37, P.1, P.1.1, P.1.2, P.2 or P3. In one embodiment, the SARSCoV-2 variant comprises at least one mutation which increases transmissibility, increases virulence of decreases vaccine efficacy, relative to the wildtype virus. In specific embodiments of the invention, the SARS-CoV-2 variant comprises at least one mutation selected from the group consisting of L18F, K417N, K417T, N439K, L452R, S477N, E484K, S494P, N501Y, D614G, P681H and P681R.

As used herein, treatment or prevention of an infection with SARS-CoV-2 refers to one or more of: (a) decreasing one or more SARS-CoV-2-related disorders; and (b) decreasing one or more SARS-CoV-2-related symptoms in a subject.

A “SARS-CoV-2-related disorder”, is a disorder that results from infection of a subject by SARS-CoV-2. SARS-CoV-2-related disorders include, but are not limited to Covid-19, respiratory illness, pneumonia, and symptoms and/or complications arising from any of these disorders.

A “SARS-CoV-2-related symptom” or a “symptom of SARS-CoV-2 infection” includes one or more physical dysfunctions related to SARS-CoV-2 infection. SARS-CoV-2 symptoms and complications include, but are not limited to, fever, dry cough, tiredness, difficulty in breathing or shortness of breath, chest pain or pressure, ageusia, parageusia, hypogeusia, anosmia, parosmia, hyposmia, and the like.

The inventors have found that Boswellia extract exerts its beneficial effects by a balanced downregulation of the immune system of a subject. Generally, Boswellia extract downregulates the expression of pro-inflammatory cytokines. Specifically, Boswellia extract was shown to inhibit activation of NF-κB (nuclear factor kappa-light-chain-enhancer of activated B cells), to downregulate TNFα (tumor necrosis factor alpha) and decrease the level of IL-1 (interleukin 1), IL-2, IL-4, IL-6, IL-8, IL-12 and IFN-γ (interferon gamma), to inhibit iNOS (nitric oxide synthase), to inhibit the formation of oxygen radicals and activation of proteases (e.g., elastase), to inhibit COX-2 (cyclooxygenase-2) and to inhibit 5-LOX (5-lipoxygenase). Thus, in one embodiment, the Boswellia extract or pharmaceutical composition inhibits activation of NF-κB, downregulates TNFα, decreases the level of IL-1, IL-2, IL-4, IL-6, IL-8, IL-12 and/or IFN-γ, inhibits iNOS, inhibits the formation of oxygen radicals and activation of proteases, inhibits COX-2 and/or inhibits 5-LOX. In one embodiment, the Boswellia extract or pharmaceutical composition inhibits activation of NF-κB in a subject. In a further embodiment, the Boswellia extract or pharmaceutical composition downregulates TNFα and decreases the level of IL-1, IL-2, IL-4, IL-6, IL-8, IL-12 and IFN-γ in a subject. In a further embodiment, the Boswellia extract or pharmaceutical composition inhibits iNOS (nitric oxide synthase) in a subject. In a further embodiment, the Boswellia extract or pharmaceutical composition inhibits the formation of oxygen radicals and activation of proteases (e.g., elastase) in a subject. In a further embodiment, the Boswellia extract or pharmaceutical composition inhibits COX-2 (cyclooxygenase-2) in a subject. In a further embodiment, the Boswellia extract or pharmaceutical composition inhibits 5-LOX (5-lipoxygenase) in a subject.

In preferred embodiments of the invention, the Boswellia extract or pharmaceutical composition inhibits activation of NF-κB, downregulates TNFα and decreases the level of IL-1, IL-2, IL-4, IL-6, IL-8, IL-12 and IFN-γ in a subject. In a further preferred embodiment, the Boswellia extract or pharmaceutical composition inhibits activation of NF-κB and iNOS (nitric oxide synthase) in a subject. In a further preferred embodiment, the Boswellia extract or pharmaceutical composition inhibits activation of NF-κB and the formation of oxygen radicals and activation of proteases (e.g., elastase) in a subject. In a further preferred embodiment, the Boswellia extract or pharmaceutical composition inhibits activation of NF-κB and COX-2 (cyclooxygenase-2) in a subject. In a further preferred embodiment, the Boswellia extract or pharmaceutical composition inhibits activation of NF-κB and 5-LOX (5-lipoxygenase) in a subject.

In yet further preferred embodiments of the invention, the Boswellia extract or pharmaceutical composition inhibits downregulates TNFα, decreases the level of IL-1, IL-2, IL-4, IL-6, IL-8, IL-12 and IFN-γ and inhibits 5-LOX (5-lipoxygenase) in a subject. In a further preferred embodiment, the Boswellia extract or pharmaceutical composition inhibits iNOS (nitric oxide synthase) and 5-LOX (5-lipoxygenase) in a subject. In a further preferred embodiment, the Boswellia extract or pharmaceutical composition inhibits the formation of oxygen radicals and activation of proteases (e.g., elastase) and 5-LOX (5-lipoxygenase) in a subject. In a further preferred embodiment, the Boswellia extract or pharmaceutical composition inhibits COX-2 (cyclooxygenase-2) and 5-LOX (5-lipoxygenase) in a subject.

SARS-CoV-2 infection has been shown to specifically stimulate IL-6 and IL-8 production. Boswellia extract and the active agents comprised therein efficiently decrease the level of IL-6 and IL-8 thus suppressing the SARS-CoV-2 immune stimulation (e.g., cytokine storm). Moreover, critical COVID-19 is associated with higher levels of IL-5, IL-6, IL-7, IL-8, IL-13, IL-15, IL-18, TNF-α, CCL2 (chemokine (C—C motif) ligand 2), CCL3, CCL4. Boswellia extract and the active agents comprised therein have been shown to inhibit the production of at least IL-6, IL-8 and TNF-α, thus suppressing the SARS-CoV-2 immune stimulation (e.g., cytokine storm).

As used herein, “cytokine storm” (also referred to as hypercytokinemia) refers to a physiological reaction in a subject (e.g., a human) in which the innate immune system causes an uncontrolled and excessive release of pro-inflammatory signalling molecules, in particular cytokines. In the context of the invention, “cytokine storm” in particular refers to an excessive release of pro-inflammatory signalling molecules due to SARS-CoV-2 infection and the associated medical conditions (i.e., SARS-CoV2-related disorders and/or SARS-CoV-2-related symptoms).

Thus, in one embodiment, the Boswellia extract or the active agents comprised therein or the pharmaceutical composition prevent, suppress, alleviate or reduce cytokine storm in a subject. In a specific embodiment, the Boswellia extract or the active agents comprised therein or the pharmaceutical composition supress the expression, or reduce the level of IL-6, IL-8 and TNFα. In another embodiment, the Boswellia extract or the active agents comprised therein supress the expression, or reduce the level of IL-6 and IL-8. In another embodiment, the Boswellia extract or the active agents comprised therein supress the expression, or reduce the level of IL-6 and TNF-α. In another embodiment, the Boswellia extract or the active agents comprised therein supress the expression, or reduce the level of IL-8 and TNF-α. In another embodiment, the Boswellia extract or the active agents comprised therein supress the expression, or reduce the level of IL-6. In another embodiment, the Boswellia extract or the active agents comprised therein supress the expression, or reduce the level of IL-8. In another embodiment, the Boswellia extract or the active agents comprised therein supress the expression, or reduce the level TNF-α.

The beneficial effects of Boswellia extract include suppression of monocyte and neutrophil activation. In one embodiment, the Boswellia extract or the active agents comprised therein suppress activation of monocytes and neutrophils. In one embodiment, the Boswellia extract or the active agents comprised therein suppress activation of monocytes. In one embodiment, the Boswellia extract or the active agents comprised therein suppress activation of neutrophils.

One advantage of the invention is that the use of the Boswellia extract according to the invention may reduce the use or dose of glucocorticoids or non-steroidal anti-inflammatory drugs (NSAIDs, e.g., cyclooxygenase inhibitors) in a subject and may shorten the duration of their use, which lead to significant side effects due to their non-specific blockade of the synthesis of all prostaglandins or possible genomic effects.

The terms “patient” and “subject” are used interchangeably and include human and non-human animal subjects, preferably human subjects, in particular those with formally diagnosed disorders SARS-CoV-2 infection/COVID-19.

Severity of SARS-CoV-2 infection can vary considerably. Subjects can generally be classified as asymptomatic COVID-19, non-severe COVID-19, severe COVID-19 and critical COVID-19 cases.

As used herein, “critical COVID-19”, or “critical SARS-CoV-2 infection” refers to confirmed SARS-CoV-2 infection with acute respiratory distress syndrome (ARDS), sepsis, septic shock, or other conditions that would normally require the provision of life-sustaining therapies such as mechanical ventilation (invasive or non-invasive) or vasopressor therapy.

As used herein, “severe COVID-19”, or “severe SARS-CoV-2 infection” refers to confirmed SARS-CoV-2 infection accompanied by any of (i) oxygen saturation <90% on room air; (ii) respiratory rate >30 breaths/min in adults and children >5 years old; ≥60 breaths/min in children <2 months old; ≥50 in children 2-11 months old; and ≥40 in children 1-5 years old; (iii) signs of severe respiratory distress (accessory muscle use, inability to complete full sentences, and, in children, very severe chest wall indrawing, grunting, central cyanosis, or presence of any other general danger signs).

As used herein, “non-severe COVID-19”, or “non-severe SARS-CoV-2 infection” refers to confirmed SARS-CoV-2 infection with symptomatic disease, but with absence of any criteria for severe or critical COVID-19.

As used herein, “asymptomatic COVID-19”, or “asymptomatic SARS-CoV-2 infection” refers to confirmed SARS-CoV-2 infection with no discernible symptoms of COVID-19.

“Confirmed SARS-CoV-2 infection” refers to SARS-CoV-2 infection confirmed by at least one positive PCR test or a similar diagnostic method.

In one embodiment of the invention, the subject to be treated suffers from asymptomatic COVID-19. In another embodiment, the subject to be treated suffers from non-severe COVID-19. In another embodiment, the subject to be treated suffers from severe COVID-19. In another embodiment, the subject to be treated suffers from critical COVID-19.

Known risk factors for increased risk of developing severe or critical COVID-19 are older age (>60 years) and pre-existing conditions such as obesity (i.e., body mass index of 30 or higher), hypertension (i.e., systolic blood pressure of 130 mmHg or above, or diastolic blood pressure of 80 mmHg or above), cardiovascular disease, diabetes mellitus, chronic lung disease (e.g., asthma or chronic obstructive pulmonary disease (COPD)), cancer and cerebrovascular disease. The subject to be treated according to the present invention may exhibit one or more of said risk factors. In one embodiment, the subject is of older age (>60 years). In another embodiment, the subject suffers from preexisting obesity. In another embodiment, the subject suffers from pre-existing hypertension. In another embodiment, the subject suffers from pre-existing cardiovascular disease. In yet another embodiment, the subject suffers from pre-existing diabetes mellitus. In yet another embodiment, the subject suffers from pre-existing chronic lung disease (e.g., asthma or chronic obstructive pulmonary disease (COPD)). In yet another embodiment, the subject suffers from pre-existing cancer. In yet another embodiment, the subject suffers from pre-existing cerebrovascular disease.

In a preferred embodiment of the invention, the subject to be treated is of older age (>60 years) and suffers from preexisting obesity. In a further preferred embodiment of the invention, the subject to be treated is of older age (>60 years) and suffers from pre-existing hypertension. In a further preferred embodiment of the invention, the subject to be treated is of older age (>60 years) and suffers from pre-existing cardiovascular disease. In a further preferred embodiment of the invention, the subject to be treated is of older age (>60 years) and suffers from pre-existing diabetes mellitus. In a further preferred embodiment of the invention, the subject to be treated is of older age (>60 years) and suffers from pre-existing chronic lung disease (e.g., asthma or chronic obstructive pulmonary disease (COPD)). In a further preferred embodiment of the invention, the subject to be treated is of older age (>60 years) and suffers from pre-existing cancer. In a further preferred embodiment of the invention, the subject to be treated is of older age (>60 years) and suffers from cerebrovascular disease.

In one embodiment of the invention, the subject to be treated is genetically predisposed to be hospitalized, or to develop severe or critical COVID-19. Several genotypic variants have been associated with increased risk of SARS-CoV-2 infection, hospitalization or death due to COVID-19, or with increased risk of developing severe or critical COVID-19. These genetic variants include Reference SNP cluster IDs (rsids) rs2271616, rs10490770, rs11919389, rs1886814, rs72711165, rs912805253, rs10774671, rs1819040, rs77534576, rs2109069, rs74956615, rs4801778 or rs13050728. Subjects comprising one or more of these genetic variants may particularly benefit from the inventive Boswellia extract-based therapy.

Thus, in one embodiment of the invention, the subject to be treated carries at least one genotypic variant associated with increased risk of SARS-CoV-2 infection, hospitalization or death. The genetic variant may be selected from the group consisting of rsids rs2271616, rs10490770, rs11919389, rs1886814, rs72711165, rs912805253, rs10774671, rs1819040, rs77534576, rs2109069, rs74956615, rs4801778 or rs13050728. Preferably, the subject to be treated carries a genetic variant that is particularly associated with disease severity, selected from the group consisting of rsids rs10490770, rs1886814, rs72711165, rs10774671, rs1819040, rs77534576, rs2109069, rs74956615 or rs13050728.

In a specific embodiment, the subject to be treated carries a genetic variant according to rsid rs2271616. In a further specific embodiment, the subject to be treated carries a genetic variant according to rsid rs10490770. In a further specific embodiment, the subject to be treated carries a genetic variant according to rsid rs11919389. In a further specific embodiment, the subject to be treated carries a genetic variant according to rsid rs1886814. In a further specific embodiment, the subject to be treated carries a genetic variant according to rsid rs72711165. In a further specific embodiment, the subject to be treated carries a genetic variant according to rsid rs912805253. In a further specific embodiment, the subject to be treated carries a genetic variant according to rsid rs10774671. In a further specific embodiment, the subject to be treated carries a genetic variant according to rsid rs1819040. In a further specific embodiment, the subject to be treated carries a genetic variant according to rsid rs77534576. In a further specific embodiment, the subject to be treated carries a genetic variant according to rsid rs2109069. In a further specific embodiment, the subject to be treated carries a genetic variant according to rsid rs74956615. In a further specific embodiment, the subject to be treated carries a genetic variant according to rsid rs4801778. In a further specific embodiment, the subject to be treated carries a genetic variant according to rsid rs13050728.

In a further embodiment, the inventive use of Boswellia extract takes into consideration epigenetic alterations in the treated subject conferred by Boswellia extract and the active agents comprised therein and the impact of such epigenetic alterations on undesired inflammatory processes in the subject (e.g., cytokine storm).

A common complication in SARS-CoV-2 patients is myocarditis, i.e., inflammation around the heart muscles, which carries the risk of fatal cardiac arrest. Efficient suppression of myocarditis reduces the risk of fatal cardiac malfunction, such as severe heart failure, refractory arrhythmias, and cardiogenic shock. Thus, in some embodiments of the invention, the subject to be treated has been diagnosed with myocarditis. Myocarditis may be diagnosed by, e.g., cardiac magnetic resonance imaging. The myocarditis may be fulminant myocarditis, acute myocarditis or chronic myocarditis.

Isolated autoantibody reactivities have been identified in patients with COVID-19, including reactivities that are characteristic of systemic autoimmune diseases. Some autoantibodies, particularly neutralizing autoantibodies against type I interferons (IFNs), appear to directly contribute to COVID-19 pathophysiology by antagonizing innate antiviral responses. In particular, there is mounting evidence that SARS-CoV-2 infection leads to the generation of autoantibodies to neuronal and astrocytic epitopes, leading to neurological COVID-19 related symptoms and long-term neurologic impairment including fatigue, depression, confusion and forgetfulness.

Boswellia extract and the active agents (e.g., boswellic acids) comprised therein have been shown to not only suppress the expression of pro-inflammatory cytokines, but also to decrease the blood levels of autoantibodies in autoimmune diseases. Accordingly, in one embodiment of the invention, the Boswellia extract or pharmaceutical composition reduce the blood level of autoantibodies in the subject. Preferably, the Boswellia extract or pharmaceutical composition reduce the blood level of autoantibodies in the subject, thereby reducing neuronal COVID-19 neurological related symptoms (e.g., fatigue, depression, confusion and/or forgetfulness).

### Items of the Invention

In addition to the aforementioned, the invention encompasses the following items.
1. Boswellia extract for use in the treatment or prevention of infection with SARS-CoV-2 or a variant thereof in a subject.
2. Boswellia extract for use according to item 1, wherein the Boswellia species is selected from the group consisting of Boswellia sacra, Boswellia carterii, Boswellia frereana, Boswellia serrata (also referred to as B. thurifera) and Boswellia papyrifera.
3. Boswellia extract for use according to item 1 or 2, wherein the Boswellia species is selected from the group consisting of Boswellia sacra, Boswellia serrata and Boswellia papyrifera.
4. Boswellia extract for use according to any one of items 1 to 3, wherein the Boswellia species is Boswellia sacra.
5. Boswellia extract for use according to item 1 or 2, wherein the Boswellia species is Boswellia carterii.
6. Boswellia extract for use according to item 1 or 2, wherein the Boswellia species is Boswellia frereana.
7. Boswellia extract for use according to any one of items 1 to 3, wherein the Boswellia species is Boswellia serrata.
8. Boswellia extract for use according to any one of items 1 to 3, wherein the Boswellia species is Boswellia papyrifera.
9. Boswellia extract for use according to any one of the aforementioned items, wherein the Boswellia extract is soluble in an organic solvent, or a mixture of organic solvents.
10. Boswellia extract for use according to any one of the aforementioned items, wherein the Boswellia extract is soluble in methanol, ethanol, hexane, ethyl acetate, diethyl ether, chloroform, methylene chloride, petroleum ether, acetone, pentane, toluene, or a mixture thereof.
11. Boswellia extract for use according to any one of the aforementioned items, wherein the Boswellia extract is soluble in methanol, ethanol, ethyl acetate, and chloroform, or a mixture thereof.
12. Boswellia extract for use according to any one of the aforementioned items, wherein the Boswellia extract is soluble in methanol.
13. Boswellia extract for use according to any one of the aforementioned items, wherein the Boswellia extract is soluble in ethanol.
14. Boswellia extract for use according to any one of the aforementioned items, wherein the Boswellia extract is soluble in ethyl acetate.
15. Boswellia extract for use according to any one of the aforementioned items, wherein the Boswellia extract is soluble in chloroform.
16. Boswellia extract for use according to any one of the aforementioned items, wherein the Boswellia extract is obtained by extraction at temperatures ranging from about 5° C. to about 150° C.
17. Boswellia extract for use according to any one of the aforementioned items, wherein the Boswellia extract is obtained by extraction at temperatures ranging from about 10° C. to about 100° C.
18. Boswellia extract for use according to any one of the aforementioned items, wherein the Boswellia extract is obtained by extraction at temperatures ranging from about 15° C. to about 75° C.
19. Boswellia extract for use according to any one of the aforementioned items, wherein the Boswellia extract is obtained by extraction at temperatures ranging from about 20° C. to about 50° C.
20. Boswellia extract for use according to any one of the aforementioned items, wherein the Boswellia extract is obtained by steam distillation of the Boswellia tree oleo resin.
21. Boswellia extract for use according to any one of items 1 to 19, wherein the Boswellia extract is obtained by a) providing crude Boswellia (e.g., Boswellia serrata) gum resin, b) extracting the resin with a water-immiscible organic solvent as disclosed herein (e.g., ethyl acetate or chloroform) and discarding the insoluble gum substances separated by filtration, c) repeatedly washing the organic extract with an aqueous solution to remove the acid compounds, wherein the aqueous solution may be an acidic, basic, or a buffered pH neutral solution, d) successive washing of the organic extract with water and saline, e) evaporation of the organic layer in vacuo at elevated temperature (e.g., 60-70° C.) to give an oil residue, f) subsequent removal of the volatile components from the above-mentioned oil residue under high vacuum and high temperature conditions to yield a Boswellia extract in the form of a viscous oil.
22. Boswellia extract for use according to any one of items 1 to 19, wherein the Boswellia extract is obtained by a) preparation of alcohol or water-alcohol extract of gum-resin Boswellia (e.g., Boswellia serrata), b) distributing the alcoholic extract between an aqueous solution and a water-immiscible organic solvent as disclosed herein (e.g., ethyl acetate or chloroform), wherein the aqueous solution may be an acidic, basic, or a buffered pH neutral solution, c) separating the organic layer, followed by obtaining an oil residue by evaporating the organic layer in vacuo at elevated temperature (e.g., 60-70° C.), d) subsequent removal of the volatile components from the above-mentioned oil residue under high vacuum and high temperatures to give a Boswellia extract in the form of a viscous oil.
23. Boswellia extract for use according to any one of the aforementioned items, wherein the Boswellia extract comprises α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) and 3-O-Acetyl-11-keto-β-boswellic acid (AKBA).
24. Boswellia extract for use according to any one of the aforementioned items, wherein the Boswellia extract comprises α-boswellic acid, β-boswellic acid, 3-O-Acetyl-β-boswellic acid (AcBA), 11-Keto-β-boswellic acid (KBA) and 3-O-Acetyl-11-keto-β-boswellic acid (AKBA).
25. Boswellia extract for use according to any one of the aforementioned items, wherein the Boswellia extract comprises α-boswellic acid, β-boswellic acid, 11-Keto-β-boswellic acid (KBA) and 3-O-Acetyl-11-keto-β-boswellic acid (AKBA).
26. Boswellia extract for use according to any one of the aforementioned items, wherein the Boswellia extract comprises 11-Keto-β-boswellic acid (KBA) and 3-O-Acetyl-11-keto-β-boswellic acid (AKBA).
27. Boswellia extract for use according to any one of the aforementioned items, wherein the Boswellia extract comprises 3-O-Acetyl-11-keto-β-boswellic acid (AKBA).
28. Boswellia extract for use according to any one of the aforementioned items, wherein the Boswellia extract comprises at least about 50%, at least about 60%, at least about 70%, at least about 75%, at least about 80%, at least about 85%, at least about 90%, or at least about 95% by weight of triterpenoid acids (i.e., boswellic acid compounds).
29. Boswellia extract for use according to item 28, wherein the Boswellia extract comprises at least about 50% by weight of triterpenoid acids (i.e., boswellic acid compounds).
30. Boswellia extract for use according to item 28, wherein the Boswellia extract comprises at least about 60% by weight of triterpenoid acids (i.e., boswellic acid compounds).
31. Boswellia extract for use according to item 28, wherein the Boswellia extract comprises at least about 70% by weight of triterpenoid acids (i.e., boswellic acid compounds).
32. Boswellia extract for use according to item 28, wherein the Boswellia extract comprises at least about 75% by weight of triterpenoid acids (i.e., boswellic acid compounds).
33. Boswellia extract for use according to item 28, wherein the Boswellia extract comprises at least about 80% by weight of triterpenoid acids (i.e., boswellic acid compounds).
34. Boswellia extract for use according to item 28, wherein the Boswellia extract comprises at least about 85% by weight of triterpenoid acids (i.e., boswellic acid compounds).
35. Boswellia extract for use according to item 28, wherein the Boswellia extract comprises at least about 90% by weight of triterpenoid acids (i.e., boswellic acid compounds).
36. Boswellia extract for use according to item 28, wherein the Boswellia extract comprises at least about 95% by weight of triterpenoid acids (i.e., boswellic acid compounds).
37. Boswellia extract for use according to any one of the aforementioned items, wherein the ratio of acetylated to nonacetylated boswellic acids is >1 (i.e., above 1).
38. Boswellia extract for use according to any one items 1 to 36, wherein the ratio of acetylated to non-acetylated boswellic acids is <1 (i.e., below 1).
39. Boswellia extract for use according to any one of the aforementioned items, wherein the Boswellia extract is selectively enriched for any one of α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) or 3-O-Acetyl-11-keto-β-boswellic acid (AKBA), or a combination thereof.
40. Boswellia extract for use according to any one of the aforementioned items, wherein the Boswellia extract is selectively enriched for α-boswellic acid.
41. Boswellia extract for use according to any one of the aforementioned items, wherein the Boswellia extract is selectively enriched for β-boswellic acid.
42. Boswellia extract for use according to any one of the aforementioned items, wherein the Boswellia extract is selectively enriched for 3-O-Acetyl-α-boswellic acid.
43. Boswellia extract for use according to any one of the aforementioned items, wherein the Boswellia extract is selectively enriched for 3-O-Acetyl-11-keto-α-boswellic acid.
44. Boswellia extract for use according to any one of the aforementioned items, wherein the Boswellia extract is selectively enriched for 3-O-Acetyl-β-boswellic acid (AcBA).
45. Boswellia extract for use according to any one of the aforementioned items, wherein the Boswellia extract is selectively enriched for 11-Keto-β-boswellic acid (KBA).
46. Boswellia extract for use according to any one of the aforementioned items, wherein the Boswellia extract is selectively enriched for 3-O-Acetyl-11-keto-β-boswellic acid (AKBA).
47. Boswellia extract for use according to any one of the aforementioned items, wherein the Boswellia extract is selectively enriched for α-boswellic acid and 3-O-Acetyl-11-keto-β-boswellic acid (AKBA).
48. Boswellia extract for use according to any one of the aforementioned items, wherein the Boswellia extract is selectively enriched for S-boswellic acid and 3-O-Acetyl-11-keto-β-boswellic acid (AKBA).
49. Boswellia extract for use according to any one of the aforementioned items, wherein the Boswellia extract is selectively enriched for 3-O-Acetyl-α-boswellic acid and 3-O-Acetyl-11-keto-β-boswellic acid (AKBA).
50. Boswellia extract for use according to any one of the aforementioned items, wherein the Boswellia extract is selectively enriched for 3-O-Acetyl-β-boswellic acid (AcBA) and 3-O-Acetyl-11-keto-β-boswellic acid (AKBA).
51. Boswellia extract for use according to any one of the aforementioned items, wherein the Boswellia extract is selectively enriched for 11-Keto-β-boswellic acid (KBA) and 3-O-Acetyl-11-keto-β-boswellic acid (AKBA).
52. Boswellia extract for use according to any one of the aforementioned items, wherein any of the aforementioned active agents or combination thereof is selectively enriched at least about 2-fold, at least about 3-fold, at least about 4-fold, at least about 5-fold, at least about 10-fold, at least about 50-fold or at least about 100-fold relative to natural Boswellia extract, i.e., relative to Boswellia extract that has not undergone selective enrichment.
53. Boswellia extract for use according to any one of the aforementioned items, wherein the Boswellia extract comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) α-boswellic acid.
54. Boswellia extract for use according to any one of the aforementioned items, wherein the Boswellia extract comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) R-boswellic acid.
55. Boswellia extract for use according to any one of the aforementioned items, wherein the Boswellia extract comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 3-O-Acetyl-α-boswellic acid.
56. Boswellia extract for use according to any one of the aforementioned items, wherein the Boswellia extract comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 3-O-Acetyl-11-keto-α-boswellic acid.
57. Boswellia extract for use according to any one of the aforementioned items, wherein the Boswellia extract comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 3-O-Acetyl-β-boswellic acid (AcBA).
58. Boswellia extract for use according to any one of the aforementioned items, wherein the Boswellia extract comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 11-Keto-β-boswellic acid (KBA).
59. Boswellia extract for use according to any one of the aforementioned items, wherein the Boswellia extract comprises at least about 1% (w/w), at least about 2% (w/w), at least about 3% (w/w), at least about 4% (w/w), at least about 5% (w/w), at least about 10% (w/w), at least about 15% (w/w), at least about 20% (w/w), at least about 25% (w/w), at least about 50% (w/w) or at least about 75% (w/w) 3-O-Acetyl-11-keto-β-boswellic acid (AKBA).
60. Boswellia extract for use according to item 59, wherein the Boswellia extract comprises at least about 1% (w/w) 3-O-Acetyl-11-keto-β-boswellic acid (AKBA).
61. Boswellia extract for use according to item 59, wherein the Boswellia extract comprises at least about 2% (w/w) 3-O-Acetyl-11-keto-β-boswellic acid (AKBA).
62. Boswellia extract for use according to item 59, wherein the Boswellia extract comprises at least about 3% (w/w) 3-O-Acetyl-11-keto-β-boswellic acid (AKBA).
63. Boswellia extract for use according to item 59, wherein the Boswellia extract comprises at least about 4% (w/w) 3-O-Acetyl-11-keto-β-boswellic acid (AKBA).
64. Boswellia extract for use according to item 59, wherein the Boswellia extract comprises at least about 5% (w/w) 3-O-Acetyl-11-keto-β-boswellic acid (AKBA).
65. Boswellia extract for use according to item 59, wherein the Boswellia extract comprises at least about 10% (w/w) 3-O-Acetyl-11-keto-β-boswellic acid (AKBA).
66. Boswellia extract for use according to item 59, wherein the Boswellia extract comprises at least about 15% (w/w) 3-O-Acetyl-11-keto-β-boswellic acid (AKBA).
67. Boswellia extract for use according to item 59, wherein the Boswellia extract comprises at least about 20% (w/w) 3-O-Acetyl-11-keto-β-boswellic acid (AKBA).
68. Boswellia extract for use according to item 59, wherein the Boswellia extract comprises at least about 25% (w/w) 3-O-Acetyl-11-keto-β-boswellic acid (AKBA).
69. Boswellia extract for use according to item 59, wherein the Boswellia extract comprises at least about 50% (w/w) 3-O-Acetyl-11-keto-β-boswellic acid (AKBA).
70. Boswellia extract for use according to item 59, wherein the Boswellia extract comprises at least about 75% (w/w) 3-O-Acetyl-11-keto-β-boswellic acid (AKBA).
71. Pharmaceutical composition comprising the Boswellia extract according to any one of the aforementioned items for use in the treatment or prevention of infection with SARS-CoV-2 or a variant thereof in a subject.
72. Pharmaceutical composition according to item 71, wherein the Boswellia extract amounts for more than 80% (w/w) of all active ingredients of pharmaceutical composition; more preferably more than 85% (w/w); and most preferably for more than 90% (w/w).
73. Pharmaceutical composition according to any of items 71 and 72, wherein the composition is essentially free from glycyrrhizin.
74. Boswellia extract or pharmaceutical composition for use according to any one of the aforementioned items, wherein the extract or composition is suitable for (i.e., formulated for) administration by inhalation, topical, oral, parenteral, intravenous, intraarterial, intraperitoneal, intramuscular, subcutaneous, rectal or vaginal administration.
75. Boswellia extract or pharmaceutical composition for use according to any one of the aforementioned items, wherein the extract or composition is suitable for (i.e., formulated for) injection, preferably for intravenous or intraarterial injection or drip.
76. Boswellia extract or pharmaceutical composition for use according to any one of items 1 to 74, wherein the extract or composition is used for the treatment of the respiratory tract by nasal, bronchial or buccal administration of, for example, aerosols or sprays.
77. Boswellia extract or pharmaceutical composition for use according to any one of items 1 to 74, wherein the extract or composition is formulated as a tablet, capsule or pill.
78. Boswellia extract or pharmaceutical composition for use according to any of the aforementioned items, wherein the extract or composition is formulated for immediate release.
79. Boswellia extract or pharmaceutical composition for use according to any of the aforementioned items, wherein the extract or composition is formulated for extended release or sustained release.
80. Boswellia extract or pharmaceutical composition for use according to any of the aforementioned items, wherein the extract or composition is formulated for pulsed release.
81. Boswellia extract or pharmaceutical composition for use according to any of the aforementioned items, wherein the extract or composition is formulated to be suitable for administration via feeding tube.
82. Pharmaceutical composition for use according to item 79, wherein the formulation comprises 5% (w/w) glucose.
83. Pharmaceutical composition for use according to item 71 to
82, wherein the composition comprises lecithin, e.g., soybean lecithin, rapeseed lecithin, sunflower lecithin, egg lecithin or a mixture thereof.
84. Pharmaceutical composition for use according to item 83, wherein the composition comprises soybean lecithin.
85. Pharmaceutical composition for use according to item 83, wherein the composition comprises rapeseed lecithin.
86. Pharmaceutical composition for use according to item 83, wherein the composition comprises sunflower lecithin.
87. Pharmaceutical composition for use according to item 83, wherein the composition comprises egg lecithin.
88. Pharmaceutical composition for use according to any one of items 71 to 87, wherein the lipid and/or lecithin content in the pharmaceutical composition is at least about 20% (w/w), at least about 30% (w/w), at least about 40% (w/w), at least about 50% (w/w), at least about 60% (w/w) or at least about 70% (w/w).
89. Pharmaceutical composition for use according to any one of items 71 to 88, wherein the lipid content in the pharmaceutical composition is at least about 20% (w/w), at least about 30% (w/w), at least about 40% (w/w), at least about 50% (w/w), at least about 60% (w/w) or at least about 70% (w/w).
90. Pharmaceutical composition for use according to any one of items 71 to 89, wherein the lecithin content in the pharmaceutical composition is at least about 20% (w/w), at least about 30% (w/w), at least about 40% (w/w), at least about 50% (w/w), at least about 60% (w/w) or at least about 70% (w/w).
91. Pharmaceutical composition for use according to item 90, wherein the lecithin content in the pharmaceutical composition is at least about 20% (w/w).
92. Pharmaceutical composition for use according to item 90, wherein the lecithin content in the pharmaceutical composition is at least about 30% (w/w).
93. Pharmaceutical composition for use according to item 90, wherein the lecithin content in the pharmaceutical composition is at least about 40% (w/w).
94. Pharmaceutical composition for use according to item 90, wherein the lecithin content in the pharmaceutical composition is at least about 50% (w/w).
95. Pharmaceutical composition for use according to item 90, wherein the lecithin content in the pharmaceutical composition is at least about 60% (w/w).
96. Pharmaceutical composition for use according to item 90, wherein the lecithin content in the pharmaceutical composition is at least about 70% (w/w).
97. Boswellia extract or pharmaceutical composition for use according to any of the aforementioned items, wherein the extract or composition is administered in a dosage of about 100 to about 4000 mg Boswellia extract, preferably about 500 to about 2500 mg Boswellia extract, more preferably about 750 to about 1500 mg Boswellia extract.
98. Boswellia extract or pharmaceutical composition for use according to any of the aforementioned items, wherein the Boswellia extract or pharmaceutical composition of the invention is administered once daily.
99. Boswellia extract or pharmaceutical composition for use according to any of items 1 to 97, wherein the Boswellia extract or pharmaceutical composition of the invention is administered twice daily.
100. Boswellia extract or pharmaceutical composition for use according to any of items 1 to 97, wherein the Boswellia extract or pharmaceutical composition of the invention is administered three times daily.
101. Boswellia extract or pharmaceutical composition for use according to any of items 1 to 97, wherein the Boswellia extract or pharmaceutical composition of the invention is administered four times daily.
102. Boswellia extract or pharmaceutical composition for use according to any of items 1 to 97, wherein the Boswellia extract or pharmaceutical composition of the invention is administered continuously.
103. Boswellia extract or pharmaceutical composition for use according to any of the aforementioned items, wherein the daily dose ranges from about 100 to about 16 000 mg, about 500 to about 10 000 mg or about 750 to about 6 000 mg Boswellia extract.
104. Boswellia extract or pharmaceutical composition for use according to any of the aforementioned items, wherein the daily dose is about 250 mg Boswellia extract.
105. Boswellia extract or pharmaceutical composition for use according to any of items 1 to 103, wherein the daily dose is about 500 mg Boswellia extract.
106. Boswellia extract or pharmaceutical composition for use according to any of items 1 to 103, wherein the daily dose is about 750 mg Boswellia extract.
107. Boswellia extract or pharmaceutical composition for use according to any of items 1 to 103, wherein the daily dose is about 1 000 mg Boswellia extract.
108. Boswellia extract or pharmaceutical composition for use according to any of items 1 to 103, wherein the daily dose is about 1 500 mg Boswellia extract.
109. Boswellia extract or pharmaceutical composition for use according to any of items 1 to 103, wherein the daily dose is about 2 000 mg Boswellia extract.
110. Boswellia extract or pharmaceutical composition for use according to any of items 1 to 103, wherein the daily dose is about 2 500 mg Boswellia extract.
111. Boswellia extract or pharmaceutical composition for use according to any of items 1 to 103, wherein the daily dose is about 3 000 mg Boswellia extract.
112. Boswellia extract or pharmaceutical composition for use according to any of items 1 to 103, wherein the daily dose is about 3 500 mg Boswellia extract.
113. Boswellia extract or pharmaceutical composition for use according to any of items 1 to 103, wherein the daily dose is about 4 000 mg Boswellia extract.
114. Boswellia extract or pharmaceutical composition for use according to any of items 1 to 103, wherein the daily dose is about 5 000 mg Boswellia extract.
115. Boswellia extract or pharmaceutical composition for use according to any of items 1 to 103, wherein the daily dose is about 6 000 mg Boswellia extract.
116. Boswellia extract or pharmaceutical composition for use according to any of the aforementioned items, wherein the Boswellia extract or pharmaceutical composition of the invention are administered via feeding tube.
117. Boswellia extract or pharmaceutical composition for use according to item 116, wherein the feeding tube is a nasogastric feeding tube.
118. Boswellia extract or pharmaceutical composition for use according to item 116, wherein the feeding tube is a nasojejunal feeding tube.
119. Boswellia extract or pharmaceutical composition for use according to item 116, wherein the feeding tube is a gastric feeding tube.
120. Boswellia extract or pharmaceutical composition for use according to item 116, wherein the feeding tube is a gastrojejunal feeding tube.
121. Boswellia extract or pharmaceutical composition for use according to item 116, wherein the feeding tube is a jejunal feeding tube.
122. Boswellia extract or pharmaceutical composition for use according to any one of items 116 to 121, wherein the Boswellia extract or pharmaceutical composition of the invention is administered continuously.
123. Boswellia extract or pharmaceutical composition for use according to any one of items 1 to 74, 78 to 80, or 83 to 115 wherein the Boswellia extract or pharmaceutical composition of the invention is administered via transdermal patch.
124. Boswellia extract or pharmaceutical composition for use according to item 123, wherein the transdermal patch comprises the Boswellia extract in an amount from about 100 to about 16 000 mg, about 500 to about 10 000 mg or about 750 to about 6 000 mg.
125. Boswellia extract or pharmaceutical composition for use according to item 123 or 124, wherein the transdermal patch comprises an amount of about 250 mg Boswellia extract.
126. Boswellia extract or pharmaceutical composition for use according to item 123 or 124, wherein the transdermal patch comprises an amount of about 500 mg Boswellia extract
127. Boswellia extract or pharmaceutical composition for use according to item 123 or 124, wherein the transdermal patch comprises an amount of about 750 mg Boswellia extract.
128. Boswellia extract or pharmaceutical composition for use according to item 123 or 124, wherein the transdermal patch comprises an amount of 1 000 mg Boswellia extract.
129. Boswellia extract or pharmaceutical composition for use according to item 123 or 124, wherein the transdermal patch comprises an amount of about 1 500 mg Boswellia extract.
130. Boswellia extract or pharmaceutical composition for use according to item 123 or 124, wherein the transdermal patch comprises an amount of about 2 000 mg Boswellia extract.
131. Boswellia extract or pharmaceutical composition for use according to item 123 or 124, wherein the transdermal patch comprises an amount of about 2 500 mg Boswellia extract.
132. Boswellia extract or pharmaceutical composition for use according to item 123 or 124, wherein the transdermal patch comprises an amount of about 3 000 mg Boswellia extract.
133. Boswellia extract or pharmaceutical composition for use according to item 123 or 124, wherein the transdermal patch comprises an amount of 3 500 mg Boswellia extract.
134. Boswellia extract or pharmaceutical composition for use according to item 123 or 124, wherein the transdermal patch comprises an amount of 4 000 mg Boswellia extract.
135. Boswellia extract or pharmaceutical composition for use according to item 123 or 124, wherein the transdermal patch comprises an amount of 5 000 mg Boswellia extract.
136. Boswellia extract or pharmaceutical composition for use according to item 123 or 124, wherein the transdermal patch comprises an amount of about 6 000 mg Boswellia extract.
137. Boswellia extract or pharmaceutical composition for use according to any one of items 123 to 136, wherein the transdermal patch is a single-layer drug-in-adhesive type patch.
138. Boswellia extract or pharmaceutical composition for use according to any one of items 123 to 136, wherein the transdermal patch is a multi-layer drug-in-adhesive type patch.
139. Boswellia extract or pharmaceutical composition for use according to any one of items 123 to 136, wherein the transdermal patch is a reservoir type patch.
140. Boswellia extract or pharmaceutical composition for use according to any one of items 123 to 139, wherein the transdermal patch has a surface area of about 10 cm², about 15 cm², about 20 cm², about 25 cm², about 30 cm², about 35 cm², about 40 cm², about 45 cm², about 50 cm², about 60 cm², about 70 cm², about 80 cm², about 90 cm², about 100 cm², about 150 cm², about 200 cm², about 300 cm², about 400 cm², about 500 cm², about 600 cm² or about 700 cm².
141. Boswellia extract or pharmaceutical composition for use according to item 140, wherein the transdermal patch has a surface area of about 100 cm², about 150 cm², about 200 cm², about 300 cm², about 400 cm² or about 500 cm².
142. Boswellia extract or pharmaceutical composition for use according to any one of items 123 to 141, wherein the transdermal patch has a surface area of about 100 cm².
143. Boswellia extract or pharmaceutical composition for use according to any one of items 123 to 141, wherein the transdermal patch has a surface area of about 150 cm².
144. Boswellia extract or pharmaceutical composition for use according to any one of items 123 to 141, wherein the transdermal patch has a surface area of about 200 cm².
145. Boswellia extract or pharmaceutical composition for use according to any one of items 123 to 141, wherein the transdermal patch has a surface area of about 300 cm².
146. Boswellia extract or pharmaceutical composition for use according to any one of items 123 to 141, wherein the transdermal patch has a surface area of about 400 cm².
147. Boswellia extract or pharmaceutical composition for use according to any one of items 123 to 141, wherein the transdermal patch has a surface area of about 500 cm².
148. Boswellia extract or pharmaceutical composition for use according to any one of the aforementioned items, wherein the extract or composition is administered in combination with a further active agent selected from the group consisting of Phyllanthus (e.g., Phyllanthus amarus), Ocimum sanctum, Andrographis panniculata, Withania somnifera, Tinosporia cordifolia, Emblica officinalis, or combinations thereof.
149. Boswellia extract or pharmaceutical composition for use according to item 148, wherein the extract or composition is administered in combination with an extract of Phyllanthus, preferrably with an extract of Phyllanthus amarus (e.g., Phyllanthus amarus Schumach et Thonn).
150. Boswellia extract or pharmaceutical composition for use according to any one of the aforementioned items, wherein the SARS-CoV-2 variant is selected from the group consisting of AV.1, AY.1, AY.2, A.23.1, B.1.1.7, B1.1.318, B.1.351, B.1.351.1, B.1.351.2, B.1.351.3, B.1.427, B.1.429, B.1.525, B.1.526, B.1.526.1, B.1.526.2, B.1.617, B.1.617.1, B.1.617.2, B.1.617.3, C.36.3, C.37, P.1, P.1.1, P.1.2, P.2 or P3.
151. Boswellia extract or pharmaceutical composition for use according to any one of the aforementioned items, wherein the SARS-CoV-2 variant comprises at least one mutation which increases transmissibility, increases virulence of decreases vaccine efficacy, relative to the wildtype virus.
152. Boswellia extract or pharmaceutical composition for use according to any one of the aforementioned items, wherein the SARS-CoV-2 variant comprises at least one mutation selected from the group consisting of L18F, K417N, K417T, N439K, L452R, S477N, E484K, S494P, N501Y, D614G, P681H and P681R.
153. Boswellia extract or pharmaceutical composition for use according to any one of the aforementioned items, wherein the Boswellia extract or pharmaceutical composition inhibits activation of NF-κB, downregulates TNFα, decreases the level of IL-1, IL-2, IL-4, IL-6, IL-8, IL-12 and/or IFN-γ, inhibits iNOS, inhibits the formation of oxygen radicals and activation of proteases, inhibits COX-2 and/or inhibits 5-LOX.
154. Boswellia extract or pharmaceutical composition for use according to item 153, wherein the Boswellia extract or pharmaceutical composition inhibits activation of NF-κB in a subject.
155. Boswellia extract or pharmaceutical composition for use according to item 153 or 154, wherein the Boswellia extract or pharmaceutical composition downregulates TNFα and decreases the level of IL-1, IL-2, IL-4, IL-6, IL-8, IL-12 and IFN-γ in a subject.
156. Boswellia extract or pharmaceutical composition for use according to any one of items 153 to 155, wherein the Boswellia extract or pharmaceutical composition inhibits iNOS (nitric oxide synthase) in a subject.
157. Boswellia extract or pharmaceutical composition for use according to any one of items 153 to 156, wherein the Boswellia extract or pharmaceutical composition inhibits the formation of oxygen radicals and activation of proteases (e.g., elastase) in a subject.
158. Boswellia extract or pharmaceutical composition for use according to any one of items 153 to 157, wherein the Boswellia extract or pharmaceutical composition inhibits COX-2 (cyclooxygenase-2) in a subject.
159. Boswellia extract or pharmaceutical composition for use according to any one of items 153 to 158, wherein the Boswellia extract or pharmaceutical composition inhibits 5-LOX (5-lipoxygenase) in a subject.
160. Boswellia extract or pharmaceutical composition for use according to any one of items 153 to 159, wherein the Boswellia extract or pharmaceutical composition inhibits activation of NF-κB, downregulates TNFα and decreases the level of IL-1, IL2, IL-4, IL-6, IL-8, IL-12 and IFN-γ in a subject.
161. Boswellia extract or pharmaceutical composition for use according to any one of items 153 to 160, wherein the Boswellia extract or pharmaceutical composition inhibits activation of NF-κB and iNOS (nitric oxide synthase) in a subject.
162. Boswellia extract or pharmaceutical composition for use according to any one of items 153 to 161, wherein the Boswellia extract or pharmaceutical composition inhibits activation of NF-κB and the formation of oxygen radicals and activation of proteases (e.g., elastase) in a subject.
163. Boswellia extract or pharmaceutical composition for use according to any one of items 153 to 162, wherein the Boswellia extract or pharmaceutical composition inhibits activation of NF-κB and COX-2 (cyclooxygenase-2) in a subject.
164. Boswellia extract or pharmaceutical composition for use according to any one of items 153 to 163, wherein the Boswellia extract or pharmaceutical composition inhibits activation of NF-κB and 5-LOX (5-lipoxygenase) in a subject.
165. Boswellia extract or pharmaceutical composition for use according to any one of items 153 to 164, wherein the Boswellia extract or pharmaceutical composition inhibits downregulates TNFα, decreases the level of IL-1, IL-2, IL-4, IL-6, IL-8, IL12 and IFN-γ and inhibits 5-LOX (5-lipoxygenase) in a subject.
166. Boswellia extract or pharmaceutical composition for use according to any one of items 153 to 165, wherein the Boswellia extract or pharmaceutical composition inhibits iNOS (nitric oxide synthase) and 5-LOX (5-lipoxygenase) in a subject.
167. Boswellia extract or pharmaceutical composition for use according to any one of items 153 to 166, wherein the Boswellia extract or pharmaceutical composition inhibits the formation of oxygen radicals and activation of proteases (e.g., elastase) and 5-LOX (5-lipoxygenase) in a subject.
168. Boswellia extract or pharmaceutical composition for use according to any one of items 153 to 167, wherein the Boswellia extract or pharmaceutical composition inhibits COX-2 (cyclooxygenase-2) and 5-LOX (5-lipoxygenase) in a subject.
169. Boswellia extract or pharmaceutical composition for use according to any one of the aforementioned items, wherein the Boswellia extract or the active agents comprised therein or the pharmaceutical composition prevent, suppress, alleviate or reduce cytokine storm in a subject.
170. Boswellia extract or pharmaceutical composition for use according to any one of the aforementioned items, wherein the Boswellia extract or the active agents comprised therein or the pharmaceutical composition supress the expression, or reduce the level of IL-6, IL-8 and TNF-α.
171. Boswellia extract or pharmaceutical composition for use according to any one of the aforementioned items, wherein the Boswellia extract or the active agents comprised therein supress the expression, or reduce the level of IL-6 and IL-8.
172. Boswellia extract or pharmaceutical composition for use according to any one of the aforementioned items, wherein the Boswellia extract or the active agents comprised therein supress the expression, or reduce the level of IL-6 and TNF-α.
173. Boswellia extract or pharmaceutical composition for use according to any one of the aforementioned items, wherein the Boswellia extract or the active agents comprised therein supress the expression, or reduce the level of IL-8 and TNF-α.
174. Boswellia extract or pharmaceutical composition for use according to any one of the aforementioned items, wherein the Boswellia extract or the active agents comprised therein supress the expression, or reduce the level of IL-6.
175. Boswellia extract or pharmaceutical composition for use according to any one of the aforementioned items, wherein the Boswellia extract or the active agents comprised therein supress the expression, or reduce the level of IL-8.
176. Boswellia extract or pharmaceutical composition for use according to any one of the aforementioned items, wherein the Boswellia extract or the active agents comprised therein supress the expression, or reduce the level TNF-α.
177. Boswellia extract or pharmaceutical composition for use according to any one of the aforementioned items, wherein the Boswellia extract or the active agents comprised therein suppress activation of monocytes and neutrophils.
178. Boswellia extract or pharmaceutical composition for use according to any one of the aforementioned items, wherein the Boswellia extract or the active agents comprised therein suppress activation of monocytes.
179. Boswellia extract or pharmaceutical composition for use according to any one of the aforementioned items, wherein the Boswellia extract or the active agents comprised therein suppress activation of neutrophils.
180. Boswellia extract or pharmaceutical composition for use according to any one of the aforementioned items, wherein the subject to be treated suffers from asymptomatic COVID-19.
181. Boswellia extract or pharmaceutical composition for use according to any one of the aforementioned items, wherein the subject to be treated suffers from non-severe COVID-19.
182. Boswellia extract or pharmaceutical composition for use according to any one of the aforementioned items, wherein the subject to be treated suffers from severe COVID-19.
183. Boswellia extract or pharmaceutical composition for use according to any one of the aforementioned items, wherein the subject to be treated suffers from critical COVID-19.
184. Boswellia extract or pharmaceutical composition for use according to any one of the aforementioned items, wherein the subject to be treated according to the present invention exhibits one or more risk factor selected from the group consisting of older age (>60 years) and pre-existing conditions such as obesity (i.e., body mass index of 30 or higher), hypertension (i.e., systolic blood pressure of 130 mmHg or above, or diastolic blood pressure of 80 mmHg or above), cardiovascular disease, diabetes mellitus, chronic lung disease (e.g., asthma or chronic obstructive pulmonary disease (COPD)), cancer and cerebrovascular disease.
185. Boswellia extract or pharmaceutical composition for use according to item 184, wherein the subject is of older age (>60 years).
186. Boswellia extract or pharmaceutical composition for use according to item 184 or 185, wherein the subject suffers from pre-existing obesity.
187. Boswellia extract or pharmaceutical composition for use according to any one of items 184 to 186, wherein the subject suffers from pre-existing hypertension.
188. Boswellia extract or pharmaceutical composition for use according to any one of items 184 to 187, wherein the subject suffers from pre-existing cardiovascular disease.
189. Boswellia extract or pharmaceutical composition for use according to any one of items 184 to 188, wherein the subject suffers from pre-existing diabetes mellitus.
190. Boswellia extract or pharmaceutical composition for use according to any one of items 184 to 189, wherein the subject suffers from pre-existing chronic lung disease (e.g., asthma or chronic obstructive pulmonary disease (COPD)).
191. Boswellia extract or pharmaceutical composition for use according to any one of items 184 to 190, wherein the subject suffers from pre-existing cancer.
192. Boswellia extract or pharmaceutical composition for use according to any one of items 184 to 191, the subject suffers from pre-existing cerebrovascular disease.
193. Boswellia extract or pharmaceutical composition for use according to any one of the aforementioned items, wherein the subject to be treated carries at least one genotypic variant associated with increased risk of SARS-CoV-2 infection, hospitalization or death.
194. Boswellia extract or pharmaceutical composition for use according to item 193, wherein the genetic variant is selected from the group consisting of rsids rs2271616, rs10490770, rs11919389, rs1886814, rs72711165, rs912805253, rs10774671, rs1819040, rs77534576, rs2109069, rs74956615, rs4801778 or rs13050728, or a combination thereof.
195. Boswellia extract or pharmaceutical composition for use according to item 194, wherein the genetic variant is selected from the group consisting of rsids rs10490770, rs1886814, rs72711165, rs10774671, rs1819040, rs77534576, rs2109069, rs74956615 or rs13050728, or a combination thereof.
196. Boswellia extract or pharmaceutical composition for use according to item 194, wherein the genetic variant is the subject to be treated carries a genetic variant according to rsid rs2271616.
197. Boswellia extract or pharmaceutical composition for use according to item 194 or 196, wherein the subject to be treated carries a genetic variant according to rsid rs10490770.
198. Boswellia extract or pharmaceutical composition for use according to item 194, 196 or 197, wherein the subject to be treated carries a genetic variant according to rsid rs11919389.
199. Boswellia extract or pharmaceutical composition for use according to item 194 or 196 to 198, wherein the subject to be treated carries a genetic variant according to rsid rs1886814.
200. Boswellia extract or pharmaceutical composition for use according to item 194 or 196 to 199, wherein the subject to be treated carries a genetic variant according to rsid rs72711165.
201. Boswellia extract or pharmaceutical composition for use according to item 194 or 196 to 200, wherein the subject to be treated carries a genetic variant according to rsid rs912805253.
202. Boswellia extract or pharmaceutical composition for use according to item 194 or 196 to 201, wherein the subject to be treated carries a genetic variant according to rsid rs10774671.
203. Boswellia extract or pharmaceutical composition for use according to item 194 or 196 to 202, wherein the subject to be treated carries a genetic variant according to rsid rs1819040.
204. Boswellia extract or pharmaceutical composition for use according to item 194 or 196 to 203, wherein the subject to be treated carries a genetic variant according to rsid rs77534576.
205. Boswellia extract or pharmaceutical composition for use according to item 194 or 196 to 204, wherein the subject to be treated carries a genetic variant according to rsid rs2109069.
206. Boswellia extract or pharmaceutical composition for use according to item 194 or 196 to 205, wherein the subject to be treated carries a genetic variant according to rsid rs74956615.
207. Boswellia extract or pharmaceutical composition for use according to item 194 or 196 to 206, wherein the subject to be treated carries a genetic variant according to rsid rs4801778.
208. Boswellia extract or pharmaceutical composition for use according to item 194 or 196 to 207, wherein the subject to be treated carries a genetic variant according to rsid rs13050728.
209. Boswellia extract or pharmaceutical composition for use according to any one of the aforementioned items, wherein the subject to be treated has been diagnosed with myocarditis.
210. Boswellia extract or pharmaceutical composition for use according to any one of the aforementioned items, wherein the Boswellia extract or pharmaceutical composition reduce the blood level of autoantibodies in the subject.
211. Boswellia extract or pharmaceutical composition for use according to item 210, wherein the Boswellia extract or pharmaceutical composition reduce the blood level of autoantibodies in the subject, thereby reducing neuronal COVID-19 neurological related symptoms (e.g., fatigue, depression, confusion and/or forgetfulness).
212. Boswellia extract or pharmaceutical composition for use according to any one of the aforementioned items, wherein 3-O-Acetyl-11-keto-β-boswellic acid (AKBA) as part of the Boswellia extract or pharmaceutical composition is used as an antiinflammatory agent, in particular by inhibiting leukotriene synthesis.
213. Boswellia extract or pharmaceutical composition for use according to any one of the aforementioned items, wherein 3-O-Acetyl-11-keto-β-boswellic acid (AKBA) as part of the Boswellia extract or pharmaceutical composition is used to reduce inflammatory pain.
214. Boswellia extract or pharmaceutical composition for use according to any one of the aforementioned items, wherein 11-Keto-β-boswellic acid (KBA) as part of the Boswellia extract or pharmaceutical composition is used as an anti-inflammatory agent, in particular by inhibiting leukotriene synthesis.
215. Boswellia extract or pharmaceutical composition for use according to any one of the aforementioned items, wherein 11-Keto-β-boswellic acid (KBA) as part of the Boswellia extract or pharmaceutical composition is used to reduce inflammatory pain.
216. Boswellia extract or pharmaceutical composition for use according to any one of the aforementioned items, wherein 3-O-Acetyl-11-keto-β-boswellic acid (AKBA) as part of the Boswellia extract or pharmaceutical composition is used as a cytokine inhibitor.
217. Boswellia extract or pharmaceutical composition for use according to any one of the aforementioned items, wherein 3-O-Acetyl-11-keto-β-boswellic acid (AKBA) as part of the Boswellia extract or pharmaceutical composition is used as an immunomodulator.
218. Boswellia extract or pharmaceutical composition for use according to any one of the aforementioned items, wherein 3-O-Acetyl-α-boswellic acid as part of the Boswellia extract or pharmaceutical composition is used as a cytokine inhibitor.
219. Boswellia extract or pharmaceutical composition for use according to any one of the aforementioned items, wherein 3-O-Acetyl-α-boswellic acid as part of the Boswellia extract or pharmaceutical composition is used as an immunomodulator.
220. Boswellia extract or pharmaceutical composition for use according to any one of the aforementioned items, wherein 11-Keto-β-boswellic acid (KBA) as part of the Boswellia extract or pharmaceutical composition is used as a cytokine inhibitor.
221. Boswellia extract or pharmaceutical composition for use according to any one of the aforementioned items, wherein 11-Keto-β-boswellic acid (KBA) as part of the Boswellia extract or pharmaceutical composition is used as an immunomodulator.
222. Boswellia extract or pharmaceutical composition for use according to any one of the aforementioned items, wherein α-boswellic acid as part of the Boswellia extract or pharmaceutical composition is used as an anti-bacterial agent.
223. Boswellia extract or pharmaceutical composition for use according to any one of the aforementioned items, wherein β-boswellic acid as part of the Boswellia extract or pharmaceutical composition is used as an anti-bacterial agent.
224. Boswellia extract or pharmaceutical composition for use according to any one of the aforementioned items, wherein α-boswellic acid as part of the Boswellia extract or pharmaceutical composition is used to improve cognitive impairments.
225. Boswellia extract or pharmaceutical composition for use according to any one of the aforementioned items, wherein 3-O-Acetyl-11-keto-β-boswellic acid (AKBA) as part of the Boswellia extract or pharmaceutical composition is used to improve cognitive impairments.
226. Boswellia extract or pharmaceutical composition for use according to any one of the aforementioned items, wherein α-boswellic acid as part of the Boswellia extract or pharmaceutical composition is used to improves inflammatory bowel diseases.
227. Boswellia extract or pharmaceutical composition for use according to any one of the aforementioned items, wherein α-boswellic acid as part of the Boswellia extract or pharmaceutical composition is used to improve ulcerative colitis.
228. Boswellia extract or pharmaceutical composition for use according to any one of the aforementioned items, wherein α-boswellic acid as part of the Boswellia extract or pharmaceutical composition is used for its gastroprotective activities.
229. Boswellia extract or pharmaceutical composition for use according to any one of the aforementioned items, wherein 3-O-Acetyl-11-keto-β-boswellic acid (AKBA) as part of the Boswellia extract or pharmaceutical composition is used to improve inflammatory bowel diseases.
230. Boswellia extract or pharmaceutical composition for use according to any one of the aforementioned items, wherein 3-O-Acetyl-11-keto-β-boswellic acid (AKBA) as part of the Boswellia extract or pharmaceutical composition is used to improve ulcerative colitis.
231. Boswellia extract or pharmaceutical composition for use according to any one of the aforementioned items, wherein 3-O-Acetyl-11-keto-β-boswellic acid (AKBA) as part of the Boswellia extract or pharmaceutical composition is used for its gastroprotective activities.
232. Boswellia extract or pharmaceutical composition for use according to any one of the aforementioned items, wherein 3-O-Acetyl-11-keto-β-boswellic acid (AKBA) as part of the Boswellia extract or pharmaceutical composition is used for its cardioprotective activities.
233. Boswellia extract or pharmaceutical composition for use according to any one of the aforementioned items, wherein 3-O-Acetyl-11-keto-β-boswellic acid (AKBA) as part of the Boswellia extract or pharmaceutical composition is used to suppress the development of myocarditis.
234. Boswellia extract or pharmaceutical composition for use according to any one of the aforementioned items, wherein 3-O-Acetyl-11-keto-β-boswellic acid (AKBA) as part of the Boswellia extract or pharmaceutical composition is used for its neuroprotective activities.
235. Boswellia extract or pharmaceutical composition for use according to any one of the aforementioned items, wherein the Boswellia extract is selectively enriched for any single of the aforementioned boswellic acids is used for the treatment of any single or any combination of the aforementioned condition(s) associated with Covid-19.
236. Boswellia extract or pharmaceutical composition for use according to any one of the aforementioned items, wherein the Boswellia extract or pharmaceutical composition is selectively enriched for any combination of the aforementioned boswellic acids is used for the treatment of any single or any combination of the aforementioned conditions associated with Covid-19.
237. Boswellia extract or pharmaceutical composition for use according to any one of the aforementioned items, wherein active agent(s) is/are used for the treatment of conditions associated with Covid-19 that persist beyond the period of viral infection (long-term effects), particularly conditions associated with long-Covid-19, post-acute-Covid-19 or chronic post-Covid-19.
238. Boswellia extract or pharmaceutical composition for use according to item 237, wherein these long-term effects comprise cardiovascular impairments.
239. Boswellia extract or pharmaceutical composition for use according to item 237, wherein these long-term effects comprise cognitive impairments.
240. Boswellia extract or pharmaceutical composition for use according to item 237, wherein these long-term effects comprise gastrointestinal impairments.
241. Boswellia extract or pharmaceutical composition for use according to item 237, wherein these long-term effects comprise organ damage.
242. Boswellia extract or pharmaceutical composition for use according to item 241, wherein the affected organ(s) is or comprise the heart.
243. Boswellia extract or pharmaceutical composition for use according to item 241, wherein the affected organ(s) is or comprise blood vessels.
244. Boswellia extract or pharmaceutical composition for use according to item 241, wherein the affected organ(s) is or comprise the brain.
245. Boswellia extract or pharmaceutical composition for use according to item 241 wherein the affected organ(s) is or comprise the nervous system.
246. Boswellia extract or pharmaceutical composition for use according to item 241 wherein the affected organ(s) is or comprise the liver.
247. Boswellia extract or pharmaceutical composition for use according to item 243, wherein the affected organ(s) is or comprise the lung.
248. Boswellia extract or pharmaceutical composition for use according to item 241 wherein the affected organ(s) is or comprise the intestine.
249. Boswellia extract or pharmaceutical composition for use according to item 241 wherein the affected organ(s) is or comprise the kidney(s).
250. Boswellia extract or pharmaceutical composition for use according to any one of the aforementioned items, wherein the active agent(s) include cembranoids.
251. Boswellia extract or pharmaceutical composition for use according to item 250, wherein the cembranoids comprise a tetrahydrofuran structure formed by cyclization between C1 and C12 through an ether bond.
252. Boswellia extract or pharmaceutical composition for use according to item 251, wherein the cembranoid is (1S,4R,11S,12R)-1:12-epoxy-11-hydroxy-cembra-7E-en-3-one (Sacraoxide C).
253. Boswellia extract or pharmaceutical composition for use according to item 251, wherein the cembranoids comprise a trisubstituted trans-olefin at the C3/4 position of the original cembranoid structure.
254. Boswellia extract or pharmaceutical composition for use according to item 253, wherein the cembranoid(s) is/are (1R,2R,5E,9Z)-1,5,9-trimethyl-12-propan-2-yl-15-10 oxabicyclo[10.2.1]pentadeca-5,9-dien-2-ol (Incensole) and/or (1R,2R,6S,8R,13S,Z)-13-isopropyl-1,10-dimethyl-5-methylene-7,16-dioxatricyclo[11.2.1.06,8]hexadec-10-en-2-ol (Boscartin AX).
255. Boswellia extract or pharmaceutical composition for use according to item 253, wherein the cembranoids comprise an 0-Acetyl group at C11.
256. Boswellia extract or pharmaceutical composition for use according to item 255, wherein the cembranoid is [(1R,2S,5E,9E,12S)-15 1,5,9-trimethyl-12-propan-2-yl-15-oxabicyclo[10.2.1]pentadeca-5,9-dien-2-yl]acetate (Incensole acetate).
257. Boswellia extract or pharmaceutical composition for use according to item 255, wherein the cembranoids comprise a carbonyl group at the C6 and/or C9 position.
258. Boswellia extract or pharmaceutical composition for use according to item 257, wherein the cembranoid(s) is/are (1S,11S,12R)-1:12-epoxy-11-acetoxy-cembra-3E,7E-dien-6-one (Sacraoxide E) and/or 20 (1S,11S,12R)-1:12-epoxy-11β-acetoxycembra-3E,7E-dien-9-one (Sacraoxide F).
259. Boswellia extract or pharmaceutical composition for use according to item 25, wherein the cembranoids comprise a hydroxy group at the Cl and/or C5 position of the original cembranoid structure.
260. Boswellia extract or pharmaceutical composition for use according to item 259 wherein the cembranoid is (1S,5R,7E,11E)-1-isopropyl-8,12-dimethyl-4-methylenecyclotetradeca-7,11-diene-1,5-diol.
261. Boswellia extract or pharmaceutical composition for use according to any one of the aforementioned items, wherein cembranoids as part of the Boswellia extract or pharmaceutical composition is used as inflammatory agents.
262. Boswellia extract or pharmaceutical composition for use according to any one of the aforementioned items, wherein Incensole as part of the Boswellia extract or pharmaceutical composition is used to support the recovery of neurobehavioral and/or cognitive functions.
263. Boswellia extract or pharmaceutical composition for use according to any one of the aforementioned items, wherein Incensole acetate as part of the Boswellia extract or pharmaceutical composition is used to support the recovery of neurobehavioral and/or cognitive functions.
264. Boswellia extract or pharmaceutical composition for use according to any one of the aforementioned items, wherein Boscartin AX as part of the Boswellia extract or pharmaceutical composition is used for its hepaprotective activities.

1. Boswellia extract for use in the treatment or prevention of infection with SARS-CoV-2 or a variant thereof in a subject.

2. Boswellia extract for use according to claim 1, wherein the Boswellia species is selected from the group consisting of Boswellia sacra, Boswellia carterii, Boswellia frereana, Boswellia serrata (also referred to as B. thurifera) and Boswellia papyrifera.

3. Boswellia extract for use according to claim 1 or 2, wherein the Boswellia extract comprises at least one active agent, selected from the group consisting of α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) and 3-O-Acetyl-11-keto-β-boswellic acid (AKBA).

4. Boswellia extract for use according to any one of claims 1 to 3, wherein the Boswellia extract comprises at least about 50% by weight of triterpenoid acids.

5. Boswellia extract for use according to any one of claims 1 to 4, wherein the Boswellia extract is selectively enriched for any one of α-boswellic acid, β-boswellic acid, 3-O-Acetyl-α-boswellic acid, 3-O-Acetyl-11-keto-α-boswellic acid, 3-O-Acetyl-β-boswellic acid (AcBA), 11-Keto-β-boswellic acid (KBA) and 3-O-Acetyl-11-keto-β-boswellic acid (AKBA) or a combination thereof.

6. Boswellia extract for use according to any one of claims 1 to 5, wherein the Boswellia extract is selectively enriched for 3-O-Acetyl-11-keto-β-boswellic acid (AKBA).

7. Pharmaceutical composition comprising the Boswellia extract according to any one of claims 1 to 6 for use in the treatment or prevention of infection with SARS-CoV-2 or a variant thereof in a subject.

8. Boswellia extract or pharmaceutical composition for use according to any one of claims 1 to 7, wherein the Boswellia extract or pharmaceutical composition is administered via feeding tube.

9. Pharmaceutical composition for use according to claim 7 or 8, wherein the pharmaceutical composition comprises soybean lecithin, rapeseed lecithin, sunflower lecithin, egg lecithin or a mixture thereof.

10. Pharmaceutical composition for use according to any one of claims 7 to 9, wherein the lecithin content in the composition is at least about 20% (w/w).

11. Boswellia extract or pharmaceutical composition for use according to any one of claims 1 to 7, wherein the Boswellia extract or pharmaceutical composition is administered via transdermal patch.

12. Boswellia extract or pharmaceutical composition for use according to any one of claims 1 to 11, wherein the daily dose is from about 100 to about 16 000 mg Boswellia extract.

13. Boswellia extract or pharmaceutical composition for use according to any one of claims 1 to 12, wherein the subject
a. exhibits one or more risk factor(s), selected from the group consisting of older age and pre-existing conditions such as obesity, hypertension, cardiovascular disease, diabetes mellitus, chronic lung disease, cancer and cerebrovascular disease; and/or
b. carries at least one genotypic variant associated with increased risk of SARS-CoV-2 infection, hospitalization or death.

14. Boswellia extract or pharmaceutical composition for use according to any one of claims 1 to 13, wherein the Boswellia extract or pharmaceutical composition inhibits activation of NF-κB, downregulates TNFα, decreases the level of IL-1, IL-2, IL-4, IL-6, IL-8, IL-12 and/or IFN-γ, inhibits iNOS, inhibits the formation of oxygen radicals and activation of proteases, inhibits COX-2 and/or inhibits 5-LOX.

15. Boswellia extract or pharmaceutical composition for use according to any one of claims 1 to 14, wherein the Boswellia extract or pharmaceutical composition prevent, suppress, alleviate or reduce cytokine storm in a subject.
